(19) United States
(12) Patent Application Publication
(10) Pub. No.: US 2026/0117234 A1
(43) Pub. Date: Apr. 30, 2026

(54) GENE SEQUENCE CONSTRUCT USED FOR TREATMENT OF CENTRAL NERVOUS SYSTEM DISEASES

(71) Applicant: Kanglin Biotech (Hangzhou) Co., Ltd., Hangzhou (CN)

(72) Inventor: Haoquan Wu, Hangzhou (CN)

(21) Appl. No.: 19/307,695

(22) Filed: Aug. 22, 2025

Related U.S. Application Data
(63) Continuation-in-part of application No. 16/969,495, now abandoned.

(30) Foreign Application Priority Data
May 31, 2018 (CN) 201810553745.9
Jul. 5, 2018 (WO) PCT/CN2018/094686

(51) Int. Cl. C12N 15/52; A61K 31/7088; A61P 25/16; C07K 14/09; C12N 7/00; C12N 9/02; C12N 9/78; C12N 9/88; C12N 15/864; C12N 15/867

(52) U.S. Cl. CPC C12N 15/52; A61K 31/7088; A61P 25/16; C07K 14/09; C12N 7/00; C12N 9/0071; C12N 9/78; C12N 9/88; C12N 15/8645; C12N 15/867; C12Y 114/16002; C12Y 305/04016; C12Y 401/01028; C12N 2740/15032; C12N 2740/15043; C12N 2750/14132; C12N 2750/14143

(57) ABSTRACT

A gene sequence construct used for the treatment of central nervous system diseases: by means of the construction of an auto-processing expression vector, tyrosine hydroxylase (TH), GTP-cyclohydrolase I (GCH1), aromatic amino acid dopa decarboxylase (AADC), and so on may be simultaneously expressed; proteins are connected by means of an auto-processing unit (APU); the use of a viral vector to introduce the construct into a target cell may ultimately result in the high-efficiency expression of tyrosine hydroxylase (TH), GTP-cyclohydrolase I (GCH1), aromatic amino acid dopa decarboxylase (AADC), and so on having independent functions, being used in the prevention or treatment of Parkinson's disease, Alzheimer's disease and other neurodegenerative diseases.

### CROSS REFERENCE TO RELATED APPLICATIONS

This application is a continuation-in-part application of U.S. Ser. No. 16/969,495, filed Aug. 12, 2020, now abandoned, which is a National Stage filing under 35 C.F.R. § 371 of International PCT Application No. PCT/CN2019/089432, filed on May 31, 2019, which claims the priority to International PCT Application No. PCT/CN2018/094686, filed on Jul. 5, 2018, and Chinese Patent Application No. CN20180553745.9, filed on May 31, 2018. The contents of the aforesaid applications are incorporated herein by reference in their entirety.

### SEQUENCE LISTING

The instant application contains a Sequence Listing which has been submitted electronically in XML format and is hereby incorporated by reference in its entirety. Said XML copy, created on Nov. 25, 2025, is named K2046-700120_SL.xml and is 39,412 bytes in size.

### TECHNICAL FIELD

The present invention relates to the field of gene therapy, in particular, to a type of gene sequence constructs for the treatment of central nervous system diseases. The gene sequence constructs can be used to prevent or treat Parkinson's disease, Alzheimer's disease, and other neurodegenerative diseases.

### BACKGROUND

Parkinson's disease (PD) is a neurodegenerative disease, characterized by the loss of dopaminergic neurons in the substantia nigra, resulting in a decrease in midbrain dopamine levels to cause the disease. Parkinson's disease affects about 1% of the world's population over 55 years of age. As society ages, the patient population will continue to grow. The common treatment for Parkinson's disease is oral administration of dopamine's precursor-levodopa, which can to some extent relieve symptoms. However, with disease progression, levodopa treatment is unsatisfactory.

Gene therapy has unique advantages for the treatment of Parkinson's disease. By targeted delivery of dopamine synthesis genes to the striatum, dopamine can be synthesized and released in the striatum, thus making the treatment of Parkinson's disease more effective. Tyrosine is catalyzed by tyrosine hydroxylase (TH) to synthesize levodopa, and then aromatic amino acid decarboxylase (AADC) converts levodopa to dopamine. TH needs tetrahydrobiopterin as a coenzyme, and GTP-cyclohydrolase 1 (GCH1) catalyzes the synthesis of tetrahydrobiopterin, and therefore the expression of TH, AADC and GCH1 can efficiently synthesize dopamine (Azzouz M. et al., 2002. J Neurosci. 22 (23): 10302-12; Jarraya B. et al., 2009. Sci Transl Med. 1 (2): 2).

In 2014, a lentiviral vector based on equine infectious anemia virus (EIAV) carrying two internal ribosome entry sites (IRES) linking three key dopamine synthetases showed good results in a phase I/II clinical trial (Palfi S. et al., 2014. Lancet. 383 (9923): 1138-46). However, this method has several problems. For example, IRES-mediated expression of three genes often leads to imbalanced expression. Therefore, Stewart H. J. et al. replaced the IRES element with a linker peptide to produce a fusion protein containing two or more of the three enzyme activities required for dopamine synthesis (Stewart H. J. et al. 2016. Hum Gene Ther Clin Dev. 27 (3): 100-10). However, TH, AADC, and GCH1 are expressed as independent proteins under natural conditions, and the fusion protein may affect the efficiency of dopamine synthesis. Therefore, to find a new protein co-expression method, to select a suitable vector for delivery into the target cells, and to express efficiently in them, are still problems to be solved for the treatment of Parkinson's disease.

### SUMMARY

The purpose of the present invention is to address, for example, the shortcomings of the existing treatment technology, by construction of auto-processing expression vectors, to express tyrosine hydroxylase (TH), GTP-cyclohydrolase I (GCH1), aromatic amino acid dopa decarboxylase (AADC), and nervous system growth factors, etc. These proteins can be linked by an auto-processing unit (APU). Viral vectors can be used for delivery into target cells, which can result in highly efficient expression of independently functional tyrosine hydroxylase (TH), GTP-cyclohydrolase I (GCH1), and aromatic amino acid dopa decarboxylase (AADC), etc., for the treatment of neurodegenerative diseases, such as Parkinson's disease. In some embodiments, a gene sequence construct described herein is used for delivery into a target cell in vivo or ex vivo. In some embodiments, the subject has Parkinson's disease. In some embodiments, the subject is a mammal, e.g., mouse, rat, or human.

In an aspect, the present invention includes, but is not limited to, the following: a gene sequence construct for the treatment of a central nervous system disease, the construct comprising nucleotide sequences that are related to the treatment of the central nervous system disease and are linked by an auto-processing unit (APU).

In some embodiments, said nucleotide sequences related to the central nervous system disease in the gene sequence construct for the treatment of the central nervous system disease are selected from two or more (e.g., three or four) of the nucleotide sequences of tyrosine hydroxylase (TH), GTP-cyclohydrolase I (GCH1), aromatic amino acid dopa decarboxylase (AADC), and a nervous system growth factor.

In some embodiments, the nucleotide sequence of TH is a nucleotide sequence of human TH or encodes an amino acid sequence of human TH. In some embodiments, the nucleotide sequence of TH encodes an amino acid sequence of human TH comprising the amino acid sequence of SEQ ID NO: 11, or an amino acid sequence that has at least 70%, 80%, 85%, 90%, 95%, 96%, 97%, 98%, or 99% identity thereto, or differs by no more than 150, 100, 75, 50, 25, 10, 5, 2, or 1 amino acids therefrom. In some embodiments, the nucleotide sequence of TH comprises the nucleotide sequence of SEQ ID NO: 12, or a nucleotide sequence that has at least 70%, 80%, 85%, 90%, 95%, 96%, 97%, 98%, or 99% identity thereto, or differs by no more than 450, 300, 150, 75, 50, 25, 10, or 5 nucleotides therefrom.

In some embodiments, the nucleotide sequence of GCH1 is a nucleotide sequence of human GCH1 or encodes an amino acid sequence of human GCH1. In some embodiments, the nucleotide sequence of GCH1 encodes an amino acid sequence of human GCH1 comprising the amino acid sequence of SEQ ID NO: 9, or an amino acid sequence that has at least 70%, 80%, 85%, 90%, 95%, 96%, 97%, 98%, or 99% identity thereto, or differs by no more than 150, 100, 75, 50, 25, 10, 5, 2, or 1 amino acids therefrom. In some embodiments, the nucleotide sequence of GCH1 comprises the nucleotide sequence of SEQ ID NO: 10, or a nucleotide sequence that has at least 70%, 80%, 85%, 90%, 95%, 96%, 97%, 98%, or 99% identity thereto, or differs by no more than 450, 300, 150, 75, 50, 25, 10, or 5 nucleotides therefrom.

In some embodiments, the nucleotide sequence of AADC is a nucleotide sequence of human AADC or encodes an amino acid sequence of human AADC. In some embodiments, the nucleotide sequence of AADC encodes an amino acid sequence of human AADC comprising the amino acid sequence of SEQ ID NO: 7, or an amino acid sequence that has at least 70%, 80%, 85%, 90%, 95%, 96%, 97%, 98%, or 99% identity thereto, or differs by no more than 150, 100, 75, 50, 25, 10, 5, 2, or 1 amino acids therefrom. In some embodiments, the nucleotide sequence of AADC comprises the nucleotide sequence of SEQ ID NO: 8, or a nucleotide sequence that has at least 70%, 80%, 85%, 90%, 95%, 96%, 97%, 98%, or 99% identity thereto, or differs by no more than 450, 300, 150, 75, 50, 25, 10, or 5 nucleotides therefrom.

In some embodiments, the gene sequence construct comprises at least two (e.g., 3 or 4) nucleotide sequences described herein that are linked by an auto-processing unit (APU). In some embodiments, the auto-processing unit (APU) comprises an N-terminal auto-processing domain and/or a C-terminal auto-processing domain.

In some embodiments, the nervous system growth factor comprises a nerve growth factor (NGF), a brain-derived neurotrophic factor (BDNF), a neurotrophin-3 (NT-3), a neurotrophin-4/5 (NT-4/5), a neurotrophin-6 (NT-6), a ciliary neurotrophic factor (CNTF), a glial cell line-derived neurotrophic factor (GDNF) and/or a GDNF family molecule (a naturally occurring analog of GDNF, neurturin, persephin, or artemin). In some embodiments, the nervous system growth factor is a human nervous system growth factor (e.g., a human GDNF).

In some embodiments, the nucleotide sequence of GDNF encodes an amino acid sequence of human GDNF comprising the amino acid sequence of SEQ ID NO: 24, or an amino acid sequence that has at least 70%, 80%, 85%, 90%, 95%, 96%, 97%, 98%, or 99% identity thereto, or differs by no more than 150, 100, 75, 50, 25, 10, 5, 2, or 1 amino acids therefrom. In some embodiments, the nucleotide sequence of GDNF comprises the nucleotide sequence of SEQ ID NO: 25, or a nucleotide sequence that has at least 70%, 80%, 85%, 90%, 95%, 96%, 97%, 98%, or 99% identity thereto, or differs by no more than 450, 300, 150, 75, 50, 25, 10, or 5 nucleotides therefrom.

Preferably, in some embodiments, the gene sequence construct for the treatment of the central nervous system disease comprises the nucleotide sequences of tyrosine hydroxylase (TH), GTP-cyclohydrolase I (GCH1), and aromatic amino acid dopa decarboxylase (AADC). In some embodiments, the gene sequence construct comprises nucleotide sequences of human TH, human GCH1, and human AADC. In some embodiments, at least two (e.g., two or all) of the nucleotide sequences are linked by an auto-processing unit (APU). In some embodiments, the nucleotide sequences of TH and GCH1 are linked by an APU. In some embodiments, the nucleotide sequences of TH and AADC are linked by an APU. In some embodiments, the nucleotide sequences of GCH1 and AADC are linked by an APU. In some embodiments, the nucleotide sequences of TH, GCH1, and AADC are linked by APUs.

In some embodiments, the N-terminal auto-processing domain comprises Intein, B-type bacterial intein-like domain (BIL), Furin sequence, or a derivative thereof. In some embodiments, the C-terminal auto-processing domain comprises a 2A peptide or a 2A-like peptide.

Preferably, in some embodiments, the 2A peptide or 2A-like peptide comprises a 2A peptide derived from foot-and-mouth disease virus (F2A), a 2A peptide derived from porcine teschovirus virus (P2A), a 2A peptide derived from insect virus (T2A), or a 2A peptide derived from equine rhinitis virus (E2A).

In another aspect, the disclosure provides a viral vector or genome, said viral vector or genome comprising any of the above-described gene sequence constructs for the treatment of a central nervous system disease. In some embodiments, said viral vector comprises a lentiviral vector or an adeno-associated viral vector. In some embodiments, said viral genome is a lentiviral genome or an adeno-associated virus genome.

In another aspect, the disclosure provides a viral vector system comprising any of the gene sequence constructs described herein for the treatment of a central nervous system disease. In some embodiments, the vector system is a lentiviral vector system. In some embodiments, said lentiviral vector system comprises a genome comprising a gene sequence construct described herein for the treatment of a central nervous system disease, and one or more nucleotide sequences encoding the gag and pol proteins and other nucleotide sequences of essential virus packaging components. In some embodiments, the vector system is an adeno-associated virus vector system.

In another aspect, the disclosure provides a cell transduced by a viral vector or genome, or a viral vector system, as described herein. In another aspect, the disclosure provides a viral particle produced by a viral vector or genome, or a viral vector system, as described herein.

In another aspect, the disclosure provides a biological product or a composition (e.g., pharmaceutical composition) comprising any of the gene sequence constructs, viral vectors or genomes, viral vector systems, cells, or viral particles, as described herein, and its use in the treatment and/or prevention of Parkinson's disease, Alzheimer's disease and other neurodegenerative diseases, for example, in a human subject. In some embodiments, the biological product or composition comprises a pharmaceutically acceptable excipient or carrier.

In another aspect, the disclosure provides a biological product or a composition (e.g., pharmaceutical composition) comprising any of the gene sequence constructs or viral vectors described herein, and other necessary viral packaging components, to produce virus particles with a pharmaceutically acceptable carrier or a diluent to form a biological product or pharmaceutical composition, and for preparing a medicament for producing dopamine in vivo, and its use in the treatment and/or prevention of Parkinson's disease, Alzheimer's disease and other neurodegenerative diseases, for example, in a human subject.

Specifically, in some embodiments, said gene sequence construct comprises selection of two or more (e.g., two or all) of the nucleotide sequences of human tyrosine hydroxylase (TH), human GTP-cyclohydrolase I (GCH1), or human aromatic amino acid dopa decarboxylase (AADC). In some embodiments, at least two (e.g., two or three) of the nucleotide sequences are linked by an auto-processing unit (APU).

In some embodiments, the gene sequence construct described herein further comprises a linker peptide coding sequence (linker), an internal ribosome entry site (IRES), or both. In some embodiments, the gene sequence construct described herein further comprises a promoter.

For example, the following is a selected gene sequence construct comprising the nucleotide sequences of tyrosine hydroxylase (TH), GTP-cyclohydrolase I (GCH1), aromatic amino acid dopa decarboxylase (AADC), the nucleotide sequences comprising at least two gene sequences that are linked by an auto-processing unit (APU). The gene sequence construct comprises the following modes of construction:
TH_{-APU-}CH1_{-APU-}AADC; TH_{-APU-}CH1_{-other sequence-}AADC; TH_{-other sequence-}CH1_{-APU-}AADC; TH_{-APU-}AADC_{-APU-}CH1; TH_{-other sequence-}AADC_{-APU-}CH1; TH_{-APU-}AADC_{-other sequence-}CH1; CH1_{-APU-}TH_{-APU-}AADC; CH1_{-APU-}TH_{-other sequence-}AADC; CH1_{-other sequence-}TH_{-APU-}AADC; CH1_{-APU-}AADC_{-APU-}TH; CH1_{-APU-}AADC_{-other sequence-}TH; CH1_{-other sequence-}AADC_{-APU-}TH; AADC_{-APU-}TH_{-APU-}CH1; AADC_{-APU-}TH_{-other sequence-}CH1; AADC_{-other sequence-}TH_{-APU-}CH1; AADC_{-APU-}CH1_{-APU-}TH; AADC_{-APU-}CH1_{-other sequence-}TH; AADC_{-other sequence-}CH1_{-APU-}TH.

APU: auto-processing unit; other sequence: comprising a linker peptide coding sequence (linker), internal ribosome entry site (IRES), promoter, or intein.

In some embodiments, the gene sequence construct further comprises a linker peptide coding sequence. In some embodiments, the linker peptide is a GS linker, e.g., a (G₄S)n linker, n=1, 2, 3, 4, 5, or 6 (SEQ ID NO: 28). In some embodiments, the linker peptide comprises the amino acid sequence of GGGGSGGGGSGGGGS (SEQ ID NO: 17) (sometimes also referred to herein as the “GS15” linker), or an amino acid sequence having at least 70%, 80%, 85%, 90%, or 95% identity thereto, or differing by no more than 5, 4, 3, 2, or 1 amino acids therefrom. In some embodiments, the linker peptide coding sequence comprises the nucleic acid sequence of GGAGGCGGCGGCTCTGGTGGAGGCGGCAGCGGCGGCGGCGGTTCT (SEQ ID NO: 18), or a nucleotide sequence having at least 70%, 80%, 85%, 90%, or 95% identity thereto, or differing by no more than 20, 10, 5, 4, 3, 2, or 1 amino acids therefrom.

In some embodiments, said auto-processing unit (APU) in a gene sequence construct described herein encodes a 2A peptide or a 2A-like peptide. In some embodiments, the 2A peptide or 2A-like peptide comprises a 2A peptide derived from foot-and-mouth disease virus (F2A), a 2A peptide derived from porcine teschovirus virus (P2A), a 2A peptide derived from insect virus (T2A), or a 2A peptide derived from equine rhinitis virus (E2A). In some embodiments, the gene sequence construct comprises a plurality of APUs that encode two or more of the 2A peptides or 2A-like peptides described herein.

In some embodiments, the gene sequence construct comprises a promoter. In some embodiments, the promoter is a constitutive promoter or a tissue-specific promoter. In some embodiments, the constitutive promoter comprises a CMV promoter, a CBH promoter, a phosphoglycerate kinase promoter, or a thymidine kinase promoter. In some embodiments, the CMV promoter comprises the nucleotide sequence of SEQ ID NO: 3, or a nucleotide sequence that has at least 70%, 80%, 85%, 90%, 95%, 96%, 97%, 98%, or 99% identity thereto, or differs by no more than 50, 25, 10, or 5 nucleotides therefrom. In some embodiments, the CBH promoter comprises the nucleotide sequence of SEQ ID NO: 1, or a nucleotide sequence that has at least 70%, 80%, 85%, 90%, 95%, 96%, 97%, 98%, or 99% identity thereto, or differs by no more than 50, 25, 10, or 5 nucleotides therefrom.

In some embodiments, the tissue-specific promoter comprises a synapsin promoter, a CD68 promoter, a GFAP promoter, or a synthetic promoter. In some embodiments, the synapsin promoter is a synapsin I promoter. In some embodiments, the synthetic promoter is a CAG promoter, a CAGG promoter, or a CASI promoter. In some embodiments, the synapsin I promoter comprises the nucleotide sequence of SEQ ID NO: 2, or a nucleotide sequence that has at least 70%, 80%, 85%, 90%, 95%, 96%, 97%, 98%, or 99% identity thereto, or differs by no more than 50, 25, 10, or 5 nucleotides therefrom. In some embodiments, the CAG promoter comprises the nucleotide sequence of SEQ ID NO: 4, or a nucleotide sequence that has at least 70%, 80%, 85%, 90%, 95%, 96%, 97%, 98%, or 99% identity thereto, or differs by no more than 50, 25, 10, or 5 nucleotides therefrom. In some embodiments, the CAGG promoter comprises the nucleotide sequence of SEQ ID NO: 5, or a nucleotide sequence that has at least 70%, 80%, 85%, 90%, 95%, 96%, 97%, 98%, or 99% identity thereto, or differs by no more than 50, 25, 10, or 5 nucleotides therefrom. In some embodiments, the CASI promoter comprises the nucleotide sequence of SEQ ID NO: 6, or a nucleotide sequence that has at least 70%, 80%, 85%, 90%, 95%, 96%, 97%, 98%, or 99% identity thereto, or differs by no more than 50, 25, 10, or 5 nucleotides therefrom.

In another aspect, the disclosure provides a method of treating or preventing a neurodegenerative disease, comprising administering to a subject in need thereof an effective amount a viral vector, viral vector system, viral particle, cell, biological product, or pharmaceutical composition, as described herein, thereby treating or preventing the neurodegenerative disease. In some embodiments, the neurodegenerative disease is Parkinson's disease. In some embodiments, the neurodegenerative disease is Alzheimer's disease. In some embodiments, the subject is a human. In some embodiments, the viral vector, viral vector system, viral particle, cell, biological product, or pharmaceutical composition is administered to the brain of the subject. In some embodiments, the viral vector, viral vector system, viral particle, cell, biological product, or pharmaceutical composition is administered to the putamen or subthalamic nucleus (STN). In some embodiments, the administration is an intraparenchymal injection. In other embodiments, the administration is an intrathecal or cisterna magna administration.

In yet another aspect, the disclosure provides a method of producing dopamine, comprising contacting a cell with a viral vector, viral vector system, viral particle, biological product, or pharmaceutical composition, as described herein, thereby producing dopamine. In some embodiments, the cell is a human cell. In some embodiments, the cell is a neuron (e.g., a dopaminergic neuron). In some embodiments, the cell is a dopamine-producing cell. In some embodiments, the contacting occurs in vivo or ex vivo.

Compared with the existing technology, the present invention has the following advantages:

1. The present invention provides a new method for linking genes for dopamine synthesis. The experimental study in the present invention shows that the method can improve the balance of target protein expression, increase the synthesis level of dopamine and its metabolites, and can improve the therapeutic effect against Parkinson's disease.

2. The present invention determines for the first time that the expression of each protein using P2A auto-processing peptide to link the expression of each protein can eventually lead to stable and balanced expression of independently functional tyrosine hydroxylase (TH), GTP-cyclohydrolase I (GCH1), aromatic amino acid dopa decarboxylase (AADC), and a nervous system growth factor, which can provide a new strategy for the treatment and/or prevention of Parkinson's disease and other neurodegenerative diseases.

### BRIEF DESCRIPTION OF THE DRAWINGS

In order to more clearly explain the technical solutions of the examples of the present invention or in the existing technology, provided below is a brief introduction of the drawings used to describe the examples or the existing technology. Apparently, the drawings described below are only certain examples of the present invention. For those of ordinary skill in the art, they may also obtain other drawings based on these drawings without creative work.

FIG. 1 is a schematic diagram of an exemplary gene sequence construct for gene therapy of Parkinson's disease of the present invention. The coding sequences for the expression of aromatic amino acid dopa decarboxylase (AADC), GTP-cyclohydrolase I (GCH1), and tyrosine hydroxylase (TH) are included. The proteins are linked by auto-processing peptide (P2A). The genes are transcribed, translated, and can produce independent aromatic amino acid dopa decarboxylase (AADC-P2A), GTP-cyclohydrolase I (GCH1-P2A), and tyrosine hydroxylase (TH), after being processed by auto-processing peptide. P2A: porcine teschovirus virus 2A auto-processed peptide.

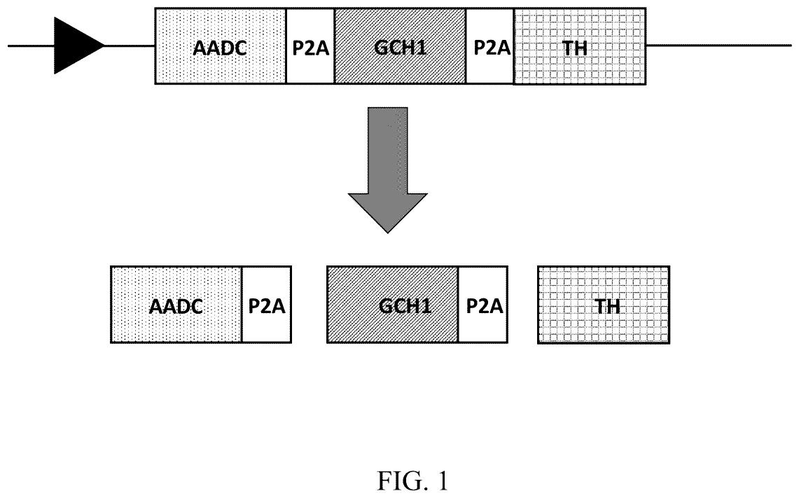

FIG. 2 is a schematic diagram of the structures of exemplary constructs of the present invention. AADC: aromatic amino acid dopa decarboxylase; GCH1: GTP-cyclohydrolase I; TH: tyrosine hydroxylase; P2A: porcine teschovirus virus 2A auto-processed peptide; Synapsin, CMV, SV40, and PGK are all promoters.

FIG. 3 shows Western blot detection results for aromatic amino acid dopa decarboxylase (AADC), GTP-cyclohydrolase I (GCH1), and tyrosine hydroxylase (TH) proteins after transduction of 293T cells. Blank: cells without viral transduction; GFP: cells transduced with CMV promoter-EGFP virus; PD-1: cells transduced with Synapsin promoter-AADC-P2A-GCH1-P2A-TH virus; PD-2: cells transduced with CMV promoter -AADC-P2A-GCH1-P2A-TH virus; P: cells transduced with CMV promoter-AADC-SV40 promoter-TH-PGK promoter-GCH1 virus; Endo-TH: endogenous TH of the cells; Exo-TH: exogenous overexpressed catalytic domain of TH.

FIG. 4 shows Western blot detection of aromatic amino acid dopa decarboxylase (AADC), GTP-cyclohydrolase I (GCH1), and tyrosine hydroxylase (TH) proteins after transduction of SH-SY5Y cells. Blank: cells without viral transduction; GFP: cells transduced with CMV promoter-EGFP virus; PD-1: cells transduced with Synapsin promoter-AADC-P2A-GCH1-P2A-TH virus; PD-2: cells transduced with CMV promoter -AADC-P2A-GCH1-P2A-TH virus; P: cells transduced with CMV promoter-AADC-SV40 promoter-TH-PGK promoter-GCH1 virus; Endo-TH: endogenous TH of the cells; Exo-TH: exogenous overexpressed catalytic domain of TH.

FIG. 5 shows the HPLC detection results of dopamine (DA) production after viral transduction of SH-SY5Y cells. GFP: cells transduced with GFP virus; PD-2: cells transduced with CMV promoter-AADC-P2A-GCH1-P2A-TH virus.

FIGS. 6A and 6B show the HPLC detection results of dopamine (DA) production after viral transduction of striatal cells. Levels of dopamine (FIG. 6A) and its metabolite homovanillic acid (FIG. 6B) were measured in striatal cells transduced with lentiviral vectors packaged with (1) EGFP expression vector as a negative control, (2) a gene sequence construct comprising AADC-P2A-GCH1-P2A-TH under the CMV promoter, or (3) the gene sequence construct comprising AADC-P2A-GCH1-P2A-TH under the synapsin promoter. Levels of dopamine and homovanillic acid were determined by HPLC. MOI=50. n=3. Mean (ng/nL)±SD are shown.

FIGS. 7A and 7B show the HPLC detection results of dopamine (DA) production after viral transduction of striatal cells. Levels of dopamine (FIG. 7A) and its metabolite homovanillic acid (FIG. 7B) were measured in striatal cells transduced with AAV vectors packaged with a gene sequence construct comprising AADC-P2A-GCH1-P2A-TH under the indicated promoters. Levels of dopamine and homovanillic acid were determined by HPLC. MOI=1E6. n=3. Mean (ng/mL)±SD are shown.

FIG. 8 shows the quantification of contralateral rotational behavior (expressed as turns/min) in a rat model of Parkinson's disease injected with AAVs comprising the gene sequence construct of AADC-P2A-GCH1-P2A-TH (DGT) or control construct (EGFP). Rats were assessed for contralateral rotational behavior induced by apomorphine beginning at four weeks after injection.

FIGS. 9A and 9B show the HPLC detection results of dopamine (DA) production after stereotactic injection in mice. Levels of dopamine (FIG. 9A) and its metabolite homovanillic acid (FIG. 9B) were measured in collected brain tissue. Levels of dopamine and homovanillic acid were determined by HPLC. MOI=1E6. n=3. Mean (ng/mL)±SD are shown.

FIG. 10 shows the dopamine (DA) concentration 72 hours after AAV-CBH-TFGPD vector infection in vitro in 293T cell supernatant. AAV9-CBH-EGFP was used as negative control. Mean (ng/mL)±SEM are shown. ***: p<0.005.

### DETAILED DESCRIPTION

### Tyrosine Hydroxylase

Exemplary amino acid and nucleotide sequences of human TH are known in the art.

An exemplary amino acid sequence of human TH is provided as follows:

(SEQ ID NO: 11)
MVKVPWFPRKVSELDKCHHLVTKFDPDLDLDHPGFSDQVYRQRRKLI
AEIAFQYRHGDPIPRVEYTAEEIATWKEVYTTLKGLYATHACGEHLE
AFALLERFSGYREDNIPQLEDVSRFLKERTGFQLRPVAGLLSARDFL
ASLAFRVFQCTQYIRHASSPMHSPEPDCCHELLGHVPMLADRTFAQF
SQDIGLASLGASDEEIEKLSTLYWFTVEFGLCKQNGEVKAYGAGLLS
SYGELLHCLSEEPEIRAFDPEAAAVQPYQDQTYQSVYFVSESFSDAK
DKLRSYASRIQRPFSVKFDPYTLAIDVLDSPQAVRRSLEGVQDELDT
LAHALSAIG

An exemplary nucleotide sequence of human TH is provided as follows:

(SEQ ID NO: 12)
ATGGTGAAGGTCCCCTGGTTCCCAAGAAAAGTGTCAGAGCTGGACAA
GTGTCATCACCTGGTCACCAAGTTCGACCCTGACCTGGACTTGGACC
ACCCGGGCTTCTCGGACCAGGTGTACCGCCAGCGCAGGAAGCTGATT
GCTGAGATCGCCTTCCAGTACAGGCACGGCGACCCGATTCCCCGTGT
GGAGTACACCGCCGAGGAGATTGCCACCTGGAAGGAGGTCTACACCA
CGCTGAAGGGCCTCTACGCCACGCACGCCTGCGGGGAGCACCTGGAG
GCCTTTGCTTTGCTGGAGCGCTTCAGCGGCTACCGGGAAGACAATAT
CCCCCAGCTGGAGGACGTCTCCCGCTTCCTGAAGGAGCGCACGGGCT
TCCAGCTGCGGCCTGTGGCCGGCCTGCTGTCCGCCCGGGACTTCCTG
GCCAGCCTGGCCTTCCGCGTGTTCCAGTGCACCCAGTATATCCGCCA
CGCGTCCTCGCCCATGCACTCCCCTGAGCCGGACTGCTGCCACGAGC
TGCTGGGGCACGTGCCCATGCTGGCCGACCGCACCTTCGCGCAGTTC
TCGCAGGACATTGGCCTGGCGTCCCTGGGGGCCTCGGATGAGGAAAT
TGAGAAGCTGTCCACGCTGTACTGGTTCACGGTGGAGTTCGGGCTGT
GTAAGCAGAACGGGGAGGTGAAGGCCTATGGTGCCGGGCTGCTGTCC
TCCTACGGGGAGCTCCTGCACTGCCTGTCTGAGGAGCCTGAGATTCG
GGCCTTCGACCCTGAGGCTGCGGCCGTGCAGCCCTACCAAGACCAGA
CGTACCAGTCAGTCTACTTCGTGTCTGAGAGCTTCAGTGACGCCAAG
GACAAGCTCAGGAGCTATGCCTCACGCATCCAGCGCCCCTTCTCCGT
GAAGTTCGACCCGTACACGCTGGCCATCGACGTGCTGGACAGCCCCC
AGGCCGTGCGGCGCTCCCTGGAGGGTGTCCAGGATGAGCTGGACACC
CTTGCCCATGCGCTGAGTGCCATTGGA

Exemplary amino acid and nucleotide sequences of human TH are also provided as Genbank Accession Nos. NP_000351.2 and NM_000360.4, respectively.

Other variant and alternative amino acid sequences of human TH are provided as Genbank Accession Nos. NP_954986.2, NP_954987.2, XP_011518637.1, and XP_054225745.1. Exemplary nucleotide sequences encoding the above-listed human TH amino acid sequences are provided as Genbank Accession No. NM_199293.3, XM_011520335.3, NM_199292.3, and XM_054369770.1, respectively.

### GTP-Cyclohydrolase I

Exemplary amino acid and nucleotide sequences of human GCH1 are known in the art.

An exemplary amino acid sequence of human GCH1 is provided as follows:

(SEQ ID NO: 9)
MEKGPVRAPAEKPRGARCSNGFPERDPPRPGPSRPAEKPPRPEAKSA
QPADGWKGERPRSEEDNELNLPNLAAAYSSILSSLGENPQRQGLLKT
PWRAASAMQFFTKGYQETISDVLNDAIFDEDHDEMVIVKDIDMFSMC
EHHLVPFVGKVHIGYLPNKQVLGLSKLARIVEIYSRRLQVQERLTKQ
IAVAITEALRPAGVGVVVEATHMCMVMRGVQKMNSKTVTSTMLGVFR
EDPKTREEFLTLIRS

An exemplary nucleotide sequence of human GCH1 is provided as follows:

(SEQ ID NO: 10)
ATGGAGAAGGGCCCTGTGCGGGCACCGGCGGAGAAGCCGCGGGGCGC
CAGGTGCAGCAATGGGTTCCCCGAGCGGGATCCGCCGCGGCCCGGGC
CCAGCAGGCCGGCGGAGAAGCCCCCGCGGCCCGAGGCCAAGAGCGCG
CAGCCCGCGGACGGCTGGAAGGGCGAGCGGCCCCGCAGCGAGGAGGA
TAACGAGCTGAACCTCCCTAACCTGGCAGCCGCCTACTCGTCCATCC
TGAGCTCGCTGGGCGAGAACCCCCAGCGGCAAGGGCTGCTCAAGACG
CCCTGGAGGGCGGCCTCGGCCATGCAGTTCTTCACCAAGGGCTACCA
GGAGACCATCTCAGATGTCCTAAACGATGCTATATTTGATGAAGATC
ATGATGAGATGGTGATTGTGAAGGACATAGACATGTTTTCCATGTGT
GAGCATCACTTGGTTCCATTTGTTGGAAAGGTCCATATTGGTTATCT
TCCTAACAAGCAAGTCCTTGGCCTCAGCAAACTTGCGAGGATTGTAG
AAATCTATAGTAGAAGACTACAAGTTCAGGAGCGCCTTACAAAACAA
ATTGCTGTAGCAATCACGGAAGCCTTGCGGCCTGCTGGAGTCGGGGT
AGTGGTTGAAGCAACACACATGTGTATGGTAATGCGAGGTGTACAGA
AAATGAACAGCAAAACTGTGACCAGCACAATGTTGGGTGTGTTCCGG
GAGGATCCAAAGACTCGGGAAGAGTTCCTGACTCTCATTAGGAGC

Exemplary amino acid and nucleotide sequences of human GCH1 are also provided as Genbank Accession Nos. NP_000152.1 and NM_000161.3, respectively.

Other variant and alternative amino acid sequences of human GCH1 are provided as Genbank Accession Nos. NP_001019195.1, NP_001019241.1, NP_001019242.1, NP_001411034.1, XP_047287217.1, XP_054231824.1, and NP_001411033.1. Exemplary nucleotide sequences encoding the above-listed human GCH1 amino acid sequences are provided as Genbank Accession No. NM_001424104.1, NM_001024071.2, NM_001024070.2, NM_001424105.1, XM_047431261.1, and NM_001024024.2, respectively.

### Aromatic Amino Acid Dopa Decarboxylase

Exemplary amino acid and nucleotide sequences of human AADC are known in the art.

An exemplary amino acid sequence of human AADC is provided as follows:

(SEQ ID NO: 7)
MNASEFRRRGKEMVDYMANYMEGIEGRQVYPDVEPGYLRPLIPAAAP
QEPDTFEDIINDVEKIIMPGVTHWHSPYFFAYFPTASSYPAMLADML
CGAIGCIGFSWAASPACTELETVMMDWLGKMLELPKAFLNEKAGEGG
GVIQGSASEATLVALLAARTKVIHRLQAASPELTQAAIMEKLVAYSS
DQAHSSVERAGLIGGVKLKAIPSDGNFAMRASALQEALERDKAAGLI
PFFMVATLGTTTCCSFDNLLEVGPICNKEDIWLHVDAAYAGSAFICP
EFRHLLNGVEFADSFNFNPHKWLLVNFDCSAMWVKKRTDLTGAFRLD
PTYLKHSHQDSGLITDYRHWQIPLGRRFRSLKMWFVFRMYGVKGLQA
YIRKHVQLSHEFESLVRQDPRFEICVEVILGLVCFRLKGSNKVNEAL
LQRINSAKKIHLVPCHLRDKFVLRFAICSRTVESAHVQRAWEHIKEL
AADVLRAERE

An exemplary nucleotide sequence of human AADC is provided as follows:

(SEQ ID NO: 8)
ATGAACGCAAGTGAATTCCGAAGGAGAGGGAAGGAGATGGTGGATTA
CATGGCCAACTACATGGAAGGCATTGAGGGACGCCAGGTCTACCCTG
ACGTGGAGCCCGGGTACCTGCGGCCGCTGATCCCTGCCGCTGCCCCT
CAGGAGCCAGACACGTTTGAGGACATCATCAACGACGTTGAGAAGAT
AATCATGCCTGGGGTGACGCACTGGCACAGCCCCTACTTCTTCGCCT
ACTTCCCCACTGCCAGCTCGTACCCGGCCATGCTTGCGGACATGCTG
TGCGGGGCCATTGGCTGCATCGGCTTCTCCTGGGCGGCAAGCCCAGC
ATGCACAGAGCTGGAGACTGTGATGATGGACTGGCTCGGGAAGATGC
TGGAACTACCAAAGGCATTTTTGAATGAGAAAGCTGGAGAAGGGGGA
GGAGTGATCCAGGGAAGTGCCAGTGAAGCCACCCTGGTGGCCCTGCT
GGCCGCTCGGACCAAAGTGATCCATCGGCTGCAGGCAGCGTCCCCAG
AGCTCACACAGGCCGCTATCATGGAGAAGCTGGTGGCTTACTCATCC
GATCAGGCACACTCCTCAGTGGAAAGAGCTGGGTTAATTGGTGGAGT
GAAATTAAAAGCCATCCCCTCAGATGGCAACTTCGCCATGCGTGCGT
CTGCCCTGCAGGAAGCCCTGGAGAGAGACAAAGCGGCTGGCCTGATT
CCTTTCTTTATGGTTGCCACCCTGGGGACCACAACATGCTGCTCCTT
TGACAATCTCTTAGAAGTCGGTCCTATCTGCAACAAGGAAGACATAT
GGCTGCACGTTGATGCAGCCTACGCAGGCAGTGCATTCATCTGCCCT
GAGTTCCGGCACCTTCTGAATGGAGTGGAGTTTGCAGATTCATTCAA
CTTTAATCCCCACAAATGGCTATTGGTGAATTTTGACTGTTCTGCCA
TGTGGGTGAAAAAGAGAACAGACTTAACGGGAGCCTTTAGACTGGAC
CCCACTTACCTGAAGCACAGCCATCAGGATTCAGGGCTTATCACTGA
CTACCGGCATTGGCAGATACCACTGGGCAGAAGATTTCGCTCTTTGA
AAATGTGGTTTGTATTTAGGATGTATGGAGTCAAAGGACTGCAGGCT
TATATCCGCAAGCATGTCCAGCTGTCCCATGAGTTTGAGTCACTGGT
GCGCCAGGATCCCCGCTTTGAAATCTGTGTGGAAGTCATTCTGGGGC
TTGTCTGCTTTCGGCTAAAGGGTTCCAACAAAGTGAATGAAGCTCTT
CTGCAAAGAATAAACAGTGCCAAAAAAATCCACTTGGTTCCATGTCA
CCTCAGGGACAAGTTTGTCCTGCGCTTTGCCATCTGTTCTCGCACGG
TGGAATCTGCCCATGTGCAGCGGGCCTGGGAACACATCAAAGAGCTG
GCGGCCGACGTGCTGCGAGCAGAGAGGGAG

Exemplary amino acid and nucleotide sequences of human AADC are also provided as Genbank Accession Nos. NP_000781.2 and NM_001082971.2, respectively.

Other variant and alternative amino acid sequences of human AADC are provided as Genbank Accession Nos. NP_001076440.2, NP_001229815.2, NP_001229816.2, NP_001229818.2, NP_001229819.2, XP_005271802.1, XP_047275887.1, XP_047275888.1, XP_054213346.1, XP_054213347.1, XP_054213348.1, and NP 001229817.2. Exemplary nucleotide sequences encoding the above-listed human AADC amino acid sequences are provided as Genbank Accession No. XM_005271745.5, XM_047419932.1, NM_001242888.2, NM_001242889.2, NM_001242887.2, NM_001082971.2, XM_047419931.1, NM_001242890.2, XM_054357372.1, XM_054357373.1, XM_054357371.1, and NM_001242886.2, respectively.

### 2A Peptides or 2A-Like Peptides

The first described auto-processing 2A peptide was derived from foot-and-mouth disease virus (FMDV). FMDV belongs to the genus Foot-and-Mouth Disease Virus of the small RNA virus family. The high-order structure of protease 2A encoded by the FMDV genome can cause steric hindrance to the center of the ribosomal peptidyl transferase, resulting in the failure to form a normal peptide chain linkage. However, at the same time the ribosome can continue to translate downstream proteins, thereby having a proteolytic enzyme-like effect, “cleaving” the two proteins in cis. Similar to FMDV, heart virus in the family of Picornaviridae, Theiler's murine encephalomyelitis virus, equine rhinitis virus, porcine teschovirus virus, etc. also contain a 2A peptide. In addition, gene sequences with similar functions of 2A peptide have been found in insect virus, type C rotavirus and trypanosome repeat sequences. The 2A peptide or 2A-like peptide auto-processing sequence, like the internal ribosomal entry site (IRES), can be used for multi-gene expression to achieve the independent expression of two or more non-fused exogenous proteins. Compared with IRES, 2A peptides or 2A-like peptides have apparent advantages in the construction of multi-gene expression vectors. For example, 2A peptides or 2A-like peptides are relatively small, and the expression of the upstream and downstream genes linked by the 2A element is well balanced. The present invention uses auto-processed peptides P2A to link TH, AADC and GCH1 to achieve independent and efficient expression of the three proteins.

Exemplary amino acid sequences of 2A peptides and 2A-like peptides are known in the art.

An exemplary amino acid sequence of F2A is provided as follows: GSGVKQTLNFDLLKLAGDVESNPGP (SEQ ID NO: 15). In some embodiments, a F2A described herein comprises the amino acid sequence of SEQ ID NO: 15, or an amino acid sequence having at least 70%, at least 80%, at least 85%, at least 90%, at least 95%, at least 96%, at least 97%, at least 98%, or at least 99% identity thereto, or an amino acid sequence differing by no more than 5, 4, 3, 2, or 1 amino acid therefrom.

An exemplary amino acid sequence of P2A is provided as follows: GSGATNFSLLKQAGDVEENPGP (SEQ ID NO: 13). In some embodiments, a P2A described herein comprises the amino acid sequence of SEQ ID NO: 13, or an amino acid sequence having at least 70%, at least 80%, at least 85%, at least 90%, at least 95%, at least 96%, at least 97%, at least 98%, or at least 99% identity thereto, or an amino acid sequence differing by no more than 5, 4, 3, 2, or 1 amino acid therefrom.

An exemplary amino acid sequence of T2A is provided as follows: GSGEGRGSLLTCGDVEENPGP (SEQ ID NO: 19). In some embodiments, a T2A described herein comprises the amino acid sequence of SEQ ID NO: 19, or an amino acid sequence having at least 70%, at least 80%, at least 85%, at least 90%, at least 95%, at least 96%, at least 97%, at least 98%, or at least 99% identity thereto, or an amino acid sequence differing by no more than 5, 4, 3, 2, or 1 amino acid therefrom.

An exemplary amino acid sequence of E2A is provided as follows: GSGQCTNYALLKLAGDVESNPGP (SEQ ID NO: 21). In some embodiments, an E2A described herein comprises the amino acid sequence of SEQ ID NO: 21, or an amino acid sequence having at least 70%, at least 80%, at least 85%, at least 90%, at least 95%, at least 96%, at least 97%, at least 98%, or at least 99% identity thereto, or an amino acid sequence differing by no more than 5, 4, 3, 2, or 1 amino acid therefrom.

In some embodiments, the F2A is encoded by a nucleic acid molecule comprising the nucleotide sequence of GGAAGCGGAGTGAAACAGACTTTGAATTTTGACCTTCTCAAGTTGGCGGGAGACGTGGA GTCCAACCCTGGACCT (SEQ ID NO: 16), or a nucleotide sequence having at least 70%, at least 80%, at least 85%, at least 90%, at least 95%, at least 96%, at least 97%, at least 98%, or at least 99% identity thereto, or a nucleotide sequence differing by no more than 25, 20, 10, 5, 4, 3, 2, or 1 nucleotide therefrom.

In some embodiments, the P2A is encoded by a nucleic acid molecule comprising the nucleotide sequence of GGAAGCGGAGCTACTAACTTCAGCCTGCTGAAGCAGGCIGGCCGACGTGGAGGAGAACCC TGGACCT (SEQ ID NO: 14), or a nucleotide sequence having at least 70%, at least 80%, at least 85%, at least 90%, at least 95%, at least 96%, at least 97%, at least 98%, or at least 99% identity thereto, or a nucleotide sequence differing by no more than 25, 20, 10, 5, 4, 3, 2, or 1 nucleotide therefrom.

In some embodiments, the T2A is encoded by a nucleic acid molecule comprising the nucleotide sequence of GGAAGCGGAGAGGGCAGAGGAAGTCTGCTAACATGCGGTGACGTCGAGGAGAATCCTG GCCCA (SEQ ID NO: 20), or a nucleotide sequence having at least 70%, at least 80%, at least 85%, at least 90%, at least 95%, at least 96%, at least 97%, at least 98%, or at least 99% identity thereto, or a nucleotide sequence differing by no more than 25, 20, 10, 5, 4, 3, 2, or 1 nucleotide therefrom.

In some embodiments, the E2A is encoded by a nucleic acid molecule comprising the nucleotide sequence of GGAAGCGGACAGTGTACTAATTATGCTCTCTTGAAATTGGCTGGAGATGTTGAGAGCAA CCCTGGACCT (SEQ ID NO: 22), or a nucleotide sequence having at least 70%, at least 80%, at least 85%, at least 90%, at least 95%, at least 96%, at least 97%, at least 98%, or at least 99% identity thereto, or a nucleotide sequence differing by no more than 25, 20, 10, 5, 4, 3, 2, or 1 nucleotide therefrom.

### Promoter

Exemplary nucleotide sequences of CMV promoter are known in the art. An exemplary nucleotide sequence of CMV promoter is provided as follows:

(SEQ ID NO: 3)
GACATTGATTATTGACTAGTTATTAATAGTAATCAATTACGGGGTCA
TTAGTTCATAGCCCATATATGGAGTTCCGCGTTACATAACTTACGGT
AAATGGCCCGCCTGGCTGACCGCCCAACGACCCCCGCCCATTGACGT
CAATAATGACGTATGTTCCCATAGTAACGCCAATAGGGACTTTCCAT
TGACGTCAATGGGTGGAGTATTTACGGTAAACTGCCCACTTGGCAGT
ACATCAAGTGTATCATATGCCAAGTACGCCCCCTATTGACGTCAATG
ACGGTAAATGGCCCGCCTGGCATTATGCCCAGTACATGACCTTATGG
GACTTTCCTACTTGGCAGTACATCTACGTATTAGTCATCGCTATTAC
CATGGTGATGCGGTTTTGGCAGTACATCAATGGGCGTGGATAGCGGT
TTGACTCACGGGGATTTCCAAGTCTCCACCCCATTGACGTCAATGGG
AGTTTGTTTTGGCACCAAAATCAACGGGACTTTCCAAAATGTCGTAA
CAACTCCGCCCCATTGACGCAAATGGGCGGTAGGCGTGTACGGTGGG
AGGTCTATATAAGCAG

Exemplary nucleotide sequences of CBH promoter are known in the art. An exemplary nucleotide sequence of CMV promoter is provided as follows:

(SEQ ID NO: 1)
CGTTACATAACTTACGGTAAATGGCCCGCCTGGCTGACCGCCCAACG
ACCCCCGCCCATTGACGTCAATAATGACGTATGTTCCCATAGTAACG
CCAATAGGGACTTTCCATTGACGTCAATGGGTGGAGTATTTACGGTA
AACTGCCCACTTGGCAGTACATCAAGTGTATCATATGCCAAGTACGC
CCCCTATTGACGTCAATGACGGTAAATGGCCCGCCTGGCATTATGCC
CAGTACATGACCTTATGGGACTTTCCTACTTGGCAGTACATCTACGT
ATTAGTCATCGCTATTACCATGGTCGAGGTGAGCCCCACGTTCTGCT
TCACTCTCCCCATCTCCCCCCCCTCCCCACCCCCAATTTTGTATTTA
TTTATTTTTTAATTATTTTGTGCAGCGATGGGGGCGGGGGGGGGGGG
GGGGCGCGCGCCAGGCGGGGCGGGGCGGGGCGAGGGGCGGGGCGGGG
CGAGGCGGAGAGGTGCGGCGGCAGCCAATCAGAGCGGCGCGCTCCGA
AAGTTTCCTTTTATGGCGAGGCGGCGGCGGCGGCGGCCCTATAAAAA
GCGAAGCGCGCGGCGGGCGGGAGTCGCTGCGACGCTGCCTTCGCCCC
GTGCCCCGCTCCGCCGCCGCCTCGCGCCGCCCGCCCCGGCTCTGACT
GACCGCGTTACTCCCACAGGTGAGCGGGCGGGACGGCCCTTCTCCTC
CGGGCTGTAATTAGCTGAGCAAGAGGTAAGGGTTTAAGGGATGGTTG
GTTGGTGGGGTATTAATGTTTAATTACCTGGAGCACCTGCCTGAAAT
CACTTTTTTTCAGGT

Exemplary nucleotide sequences of synapsin I promoter are known in the art. An exemplary nucleotide sequence of synapsin I promoter is provided as follows:

(SEQ ID NO: 2)
CGTTACATAACTTACGGTAAATGGCCCGCCTGGCTGACCGCCCAAC
GACCCCCGCCCATTGACGTCAATAATGACGTATGTTCCCATAGTAA
CGCCAATAGGGACTTTCCATTGACGTCAATGGGTGGAGTATTTACG
GTAAACTGCCCACTTGGCAGTACATCAAGTGTATCATATGCCAAGT
ACGCCCCCTATTGACGTCAATGACGGTAAATGGCCCGCCTGGCATT
ATGCCCAGTACATGACCTTATGGGACTTTCCTACTTGGCAGTACAT
CTACGTATTAGTCATCGCTATTACCATGGCTGCAGAGGGCCCTGCG
TATGAGTGCAAGTGGGTTTTAGGACCAGGATGAGGCGGGGTGGGGG
TGCCTACCTGACGACCGACCCCGACCCACTGGACAAGCACCCAACC
CCCATTCCCCAAATTGCGCATCCCCTATCAGAGAGGGGGAGGGGAA
ACAGGATGCGGCGAGGCGCGTGCGCACTGCCAGCTTCAGCACCGCG
GACAGTGCCTTCGCCCCCGCCTGGCGGCGCGCGCCACCGCCGCCTC
AGCACTGAAGGCGCGCTGACGTCACTCGCCGGTCCCCCGCAAACTC
CCCTTCCCGGCCACCTTGGTCGCGTCCGCGCCGCCGCCGGCCCAGC
CGGACCGCACCACGCGAGGCGCGAGATAGGGGGGCACGGGCGCGAC
CATCTGCGCTGCGGCG

Exemplary nucleotide sequences of CAG promoter are known in the art. An exemplary nucleotide sequence of synapsin I promoter is provided as follows:

(SEQ ID NO: 4)
CCATTGACGTCAATAATGACGTATGTTCCCATAGTAACGCCAATAG
GGACTTTCCATTGACGTCAATGGGTGGAGTATTTACGGTAAACTGC
CCACTTGGCAGTACATCAAGTGTATCATATGCCAAGTACGCCCCCT
ATTGACGTCAATGACGGTAAATGGCCCGCCTGGCATTATGCCCAGT
ACATGACCTTATGGGACTTTCCTACTTGGCAGTACATCTACGTATT
AGTCATCGCTATTACCATGGTCGAGGTGAGCCCCACGTTCTGCTTC
ACTCTCCCCATCTCCCCCCCCTCCCCACCCCCAATTTTGTATTTAT
TTATTTTTTAATTATTTTGTGCAGCGATGGGGGCGGGGGGGGGGGG
GGGGGGCGCGCCAGGCGGGGCGGGGCGGGGCGAGGGGCGGGGCGGG
GCGAGGCGGAGAGGTGCGGCGGCAGCCAATCAGAGCGGCGCGCTCC
GAAAGTTTCCTTTTATGGCGAGGCGGCGGCGGCGGCGGCCCTATAA
AAAGCGAAGCGCGCGGCGGGCGGGAGTCGCTGCGCGCTGCCTTCGC
CCCGTGCCCCGCTCCGCCGCCGCCTCGCGCCGCCCGCCCCGGCTCT
GACTGACCGCGTTACTCCCACAGGTGAGCGGGCGGGACGGCCCTTC
TCCTCCGGGCTGTAATTAGCGCTTGGTTTAATGACGGCTTGTTTCT
TTTCTGTGGCTGCGTGAAAGCCTTGAGGGGCTCCGGGAGGGCCCTT
TGTGCGGGGGGAGCGGCTCGGGGCTGTCCGCGGGGGGACGGCTGCC
TTCGGGGGGGACGGGGCAGGGCGGGGTTCGGCTTCTGGCGTGTGAC
CGGCGGCTCTAGAGCCTCTGCTAACCATGTTCATGCCTTCTTCTTT
TTCCTACAGCTCCTGGGCAACGT

Exemplary nucleotide sequences of CAGG promoter are known in the art. An exemplary nucleotide sequence of synapsin I promoter is provided as follows:

SEQ ID NO: 5
(SEQ ID NO: 5)
GTTACATAACTTACGGTAAATGGCCCGCCTGGCTGACCGCCCAACG
ACCCCCGCCCATTGACGTCAATAATGACGTATGTTCCCATAGTAAC
GCCAATAGGGACTTTCCATTGACGTCAATGGGTGGAGTATTTACGG
TAAACTGCCCACTTGGCAGTACATCAAGTGTATCATATGCCAAGTA
CGCCCCCTATTGACGTCAATGACGGTAAATGGCCCGCCTGGCATTA
TGCCCAGTACATGACCTTATGGGACTTTCCTACTTGGCAGTACATC
TACGTATTAGTCATCGCTATTACCATGGTCGAGGTGAGCCCCACGT
TCTGCTTCACTCTCCCCATCTCCCCCCCCTCCCCACCCCCAATTTT
GTATTTATTTATTTTTTAATTATTTTGTGCAGCGATGGGGGCGGGG
GGGGGGGGGGGGCGCGCGCCAGGCGGGGCGGGGCGGGGCGAGGGGC
GGGGCGGGGCGAGGCGGAGAGGTGCGGCGGCAGCCAATCAGAGCGG
CGCGCTCCGAAAGTTTCCTTTTATGGCGAGGCGGCGGCGGCGGCGG
CCCTATAAAAAGCGAAGCGCGCGGCGGGGGGGGAGTCGCTGCGACG
CTGCCTTCGCCCCGTGCCCCGCTCCGCCGCCGCCTCGCGCCGCCCG
CCCCGGCTCTGACTGACCGCGTTACTCCCACAGGTGAGCGGGCGGG
ACGGCCCTTCTCCTCCGGGCTGTAATTAGCGCTTGGTTTAATGACG
GCTTGTTTCTTTTCTGTGGCTGCGTGAAAGCCTTGAGGGGCTCCGG
GAGGGCCCTTTGTGCGGGGGGAGCGGCTCGGGGGGTGCGTGCGTGT
GTGTGTGCGTGGGGAGCGCCGCGTGCGGCTCCGCGCTGCCCGGCGG
CTGTGAGCGCTGCGGGCGCGGCGCGGGGCTTTGTGCGCTCCGCAGT
GTGCGCGAGGGGAGCGCGGCCGGGGGCGGTGCCCCGCGGTGCGGGG
GGGGCTGCGAGGGGAACAAAGGCTGCGTGCGGGGTGTGTGCGTGGG
GGGGTGAGCAGGGGGTGTGGGCGCGTCGGTCGGGCTGCAACCCCCC
CTGCACCCCCCTCCCCGAGTTGCTGAGCACGGCCCGGCTTCGGGTG
CGGGGCTCCGTACGGGGCGTGGCGCGGGGCTCGCCGTGCCGGGCGG
GGGGTGGCGGCAGGTGGGGGTGCCGGGCGGGGCGGGGCCGCCTCGG
GCCGGGGAGGGCTCGGGGGAGGGGCGCGGCGGCCCCCGGAGCGCCG
GCGGCTGTCGAGGCGCGGCGAGCCGCAGCCATTGCCTTTTATGGTA
ATCGTGCGAGAGGGCGCAGGGACTTCCTTTGTCCCAAATCTGTGCG
GAGCCGAAATCTGGGAGGCGCCGCCGCACCCCCTCTAGCGGGCGCG
GGGCGAAGCGGTGCGGCGCCGGCAGGAAGGAAATGGGCGGGGAGGG
CCTTCGTGCGTCGCCGCGCCGCCGTCCCCTTCTCCCTCTCCAGCCT
CGGGGCTGTCCGCGGGGGGACGGCTGCCTTCGGGGGGGACGGGGCA
GGGCGGGGTTCGGCTTCTGGCGTGTGACCGGCGGCTCTAGAGCCTC
TGCTAACCATGTTCATGCCTTCTTCTTTTTCCTACAG

Exemplary nucleotide sequences of CASI promoter are known in the art. An exemplary nucleotide sequence of synapsin I promoter is provided as follows:

(SEQ ID NO: 6)
GTTACATAACTTACGGTAAATGGCCCGCCTGGCTGACCGCCCAACG
ACCCCCGCCCATTGACGTCAATAATGACGTATGTTCCCATAGTAAC
GCCAATAGGGACTTTCCATTGACGTCAATGGGTGGAGTATTTACGG
TAAACTGCCCACTTGGCAGTACATCAAGTGTATCATATGCCAAGTA
CGCCCCCTATTGACGTCAATGACGGTAAATGGCCCGCCTGGCATTA
TGCCCAGTACATGACCTTATGGGACTTTCCTACTTGGCAGTACATC
TACGTATTAGTCATCGCTATTACCATGGTCGAGGTGAGCCCCACGT
TCTGCTTCACTCTCCCCATCTCCCCCCCCTCCCCACCCCCAATTTT
GTATTTATTTATTTTTTAATTATTTTGTGCAGCGATGGGGGCGGGG
GGGGGGGGGGGGCGCGCGCCAGGCGGGGCGGGGCGGGGCGAGGGGC
GGGGCGGGGCGAGGCGGAGAGGTGCGGCGGCAGCCAATCAGAGCGG
CGCGCTCCGAAAGTTTCCTTTTATGGCGAGGCGGCGGCGGCGGCGG
CCCTATAAAAAGCGAAGCGCGCGGCGGGCGGGAGTCGCTGCGCGCT
GCCTTCGCCCCGTGCCCCGCTCCGCCGCCGCCTCGCGCCGCCCGCC
CCGGCTCTGACTGACCGCGTTACTAAAACAGGTAAGTCCGGCCTCC
GCGCCGGGTTTTGGCGCCTCCCGCGGGCGCCCCCCTCCTCACGGCG
AGCGCTGCCACGTCAGACGAAGGGCGCAGCGAGCGTCCTGATCCTT
CCGCCCGGACGCTCAGGACAGCGGCCCGCTGCTCATAAGACTCGGC
CTTAGAACCCCAGTATCAGCAGAAGGACATTTTAGGACGGGACTTG
GGTGACTCTAGGGCACTGGTTTTCTTTCCAGAGAGCGGAACAGGCG
AGGAAAAGTAGTCCCTTCTCGGCGATTCTGCGGAGGGATCTCCGTG
GGGCGGTGAACGCCGATGATGCCTCTACTAACCATGTTCATGTTTT
CTTTTTTTTTCTACAGGTCCTGGGTGACGAACAGGCTAGC

### IRES

Exemplary nucleotide sequences of IRES are known in the art.

An exemplary nucleotide sequence of IRES is provided as follows:

(SEQ ID NO: 23)
CTCAAACCCGCTGATCAGCCTCGACTGTGCCTTCTAGTTGCCAGCC
ATCTGTTGTTTGCCCCTCCCCCGTGCCTTCCTTGAGAATTCCTCGA
CGTAGATATCTTAAAACAGCTCTGGGGTTGTACCCACCCCAGAGGC
CCACGTGGCGGCTAGTACTCCGGTATTGCGGTACCTTTGTACGCCT
GTTTTATACTCCCTTCCCCCGTAACTTAGAAGCACAATGTCCAAGT
TCAATAGGAGGGGGTGCAAACCAGTACCACCACGAACAAGCACTTC
TGTTCCCCCGGTGAGGCTGTATAGGCTGTTTCCACGGCTAAAAGCG
GCTGATCCGTTATCCGCTCATGTACTTCGAGAAGCCTAGTATCACC
TTGGAATCTTCGATGCGTTGCGCTCAACACTCAACCCCAGAGTGTA
GCTTAGGTCGATGAGTCTGGACGTTCCTCACCGGCGACGGTGGTCC
AGGCTGCGTTGGCGGCCTACCTGTGGCCCAAAGCCACAGGACGCTA
GTTGTGAACAAGGTGTGAAGAGCCTATTGAGCTACCTGAGAGTCCT
CCGGCCCCTGAATGCGGCTAATCCTAACCACGGAGCAGGCAGTGGC
AATCCAGCGACCAGCCTGTCGTAACGCGCAAGTTCGTGGCGGAACC
GACTACTTTGGGTGTCCGTGTTTCCTTTTATTTTTACAATGGCTGC
TTATGGTGACAATCATTGATTGTTATCATAAAGCAAATTGGATTGG
CCATCCGGTGAGAATTTGATTATTAAATTACTCTCTTGTTGGGATT
GCTCCTTTGAAATCTTGTGCACTCACACCTATTGGAATTACCTCAT
TGTTAAGATACGCGTCTAGCTAGCGCCACC

### Nervous System Growth Factor

Exemplary amino acid and nucleotide sequences of human GDNF are known in the art.

An exemplary amino acid sequence of human GDNF is provided as follows:

(SEQ ID NO: 24)
MKLWDVVAVCLVLLHTASAFPLPAGKRPPEAPAEDRSLGRRRAPFA
LSSDSNMPEDYPDQFDDVMDFIQATIKRLKRSPDKQMAVLPRRERN
RQAAAANPENSRGKGRRGQRGKNRGCVLTAIHLNVTDLGLGYETKE
ELIFRYCSGSCDAAETTYDKILKNLSRNRRLVSDKVGQACCRPIAF
DDDLSFLDDNLVYHILRKHSAKRCGCI

An exemplary nucleotide sequence encoding human GDNF is provided as follows:

(SEQ ID NO: 25)
ATGAAGTTATGGGATGTCGTGGCTGTCTGCCTGGTGCTGCTCCACA
CCGCGTCCGCCTTCCCGCTGCCCGCCGGTAAGAGGCCTCCCGAGGC
GCCCGCCGAAGACCGCTCCCTCGGCCGCCGCCGCGCGCCCTTCGCG
CTGAGCAGTGACTCAAATATGCCAGAGGATTATCCTGATCAGTTCG
ATGATGTCATGGATTTTATTCAAGCCACCATTAAAAGACTGAAAAG
GTCACCAGATAAACAAATGGCAGTGCTTCCTAGAAGAGAGCGGAAT
CGGCAGGCTGCAGCTGCCAACCCAGAGAATTCCAGAGGAAAAGGTC
GGAGAGGCCAGAGGGGCAAAAACCGGGGTTGTGTCTTAACTGCAAT
ACATTTAAATGTCACTGACTTGGGTCTGGGCTATGAAACCAAGGAG
GAACTGATTTTTAGGTACTGCAGCGGCTCTTGCGATGCAGCTGAGA
CAACGTACGACAAAATATTGAAAAACTTATCCAGAAATAGAAGGCT
GGTGAGTGACAAAGTAGGGCAGGCATGTTGCAGACCCATCGCCTTT
GATGATGACCTGTCGTTTTTAGATGATAACCTGGTTTACCATATTC
TAAGAAAGCATTCCGCTAAAAGGTGTGGATGTATCTGA

### ENUMERATED EMBODIMENTS

1. A gene sequence construct, comprising a plurality of nucleotide sequences that are related to the treatment of a central nervous system disease, and wherein two or more nucleotide sequences in the plurality of nucleotide sequences are linked by an auto-processing unit (APU).

2. The gene sequence construct of embodiment 1, wherein the plurality of nucleotide sequences comprises two or more of the nucleotide sequences of tyrosine hydroxylase (TH), GTP-cyclohydrolase I (GCH1), aromatic amino acid dopa decarboxylase (AADC), or a nervous system growth factor.

3. The gene sequence construct of embodiment 1 or 2, wherein the plurality of nucleotide sequences comprises three or all of the nucleotide sequences of TH, GCH1, AADC, or a nervous system growth factor.

4. The gene sequence construct of embodiment 2 or 3, wherein the nervous system growth factor comprises a nerve growth factor (NGF), a brain-derived neurotrophic factor (BDNF), a neurotrophin-3 (NT-3), a neurotrophin-4/5 (NT-4/5), a neurotrophin-6 (NT-6), a ciliary neurotrophic factor (CNTF), a glial cell line-derived neurotrophic factor (GDNF), or a GDNF family molecule.

5. The gene sequence construct of embodiment 4, wherein the GDNF family molecule comprises a naturally occurring analog of GDNF, neurturin, persephin, or artemin.

6. The gene sequence construct of any of embodiments 1-3, wherein the plurality of nucleotide sequences comprises the nucleotide sequences of TH, GCH1, and AADC.

7. The gene sequence construct of any of embodiments 1-6, wherein the plurality of nucleotide sequences comprises a nucleotide sequence of TH, and wherein the nucleotide sequence of TH encodes an amino acid sequence of SEQ ID NO: 11 or an amino acid sequence having at least 90% sequence identity thereto.

8. The gene sequence construct of any of embodiments 1-7, wherein the plurality of nucleotide sequences comprises a nucleotide sequence of GCH1, and wherein the nucleotide sequence of GCH1 encodes an amino acid sequence of SEQ ID NO: 9 or an amino acid sequence having at least 90% sequence identity thereto.

9. The gene sequence construct of any of embodiments 1-8, wherein the plurality of nucleotide sequences comprises a nucleotide sequence of AADC, and wherein the nucleotide sequence of TH encodes an amino acid sequence of SEQ ID NO: 7 or an amino acid sequence having at least 90% sequence identity thereto.

10. The gene sequence construct of any of embodiments 1-9, wherein the APU encodes a 2A peptide or 2A-like peptide.

11. The gene sequence construct of embodiment 10, wherein the 2A peptide or 2A-like peptide comprises a 2A peptide derived from foot-and-mouth disease virus (F2A), a 2A peptide derived from porcine teschovirus virus (P2A), a 2A peptide derived from insect virus (T2A), or a 2A peptide derived from equine rhinitis virus (E2A).

12. The gene sequence construct of any of embodiments 1-11, wherein the APU comprises an N-terminal auto-processing domain and/or a C-terminal auto-processing domain.

13. The gene sequence construct of embodiment 12, wherein the N-terminal auto-processing domain comprises Intein, B-type bacterial intein-like domain (BIL), Furin sequence, or a derivative thereof.

14. The gene sequence construct of embodiment 12 or 13, wherein the C-terminal auto-processing domain comprises a 2A peptide or a 2A-like peptide.

15. The gene sequence construct of any of the preceding embodiments, wherein the gene sequence construct comprises the nucleotide sequences of human TH, GCH1, and AADC, wherein at least two of the nucleotide sequences of human TH, GCH1, and AADC are linked by an APU.

16. The gene sequence construct of any of the preceding embodiments, characterized in that the gene sequence construct comprises the following modes of construction: TH_{-APU-}CH1_{-APU-}AADC; TH_{-APU-}CH1_{-other sequence-}AADC; TH_{-other sequence-}CH1_{-APU-}AADC; TH_{-APU-}AADC_{-APU-}CH1; TH_{-other sequence-}AADC_{-APU-}CH1; TH_{-APU-}AADC_{-other sequence-}CH1; CH1_{-APU-}TH_{-APU-}AADC; CH1_{-APU-}TH_{-other sequence-}AADC; CH1_{-other sequence-}TH_{-APU-}AADC; CH1_{-APU-}AADC_{-APU-}TH; CH1_{-APU-}AADC_{-other sequence-}TH; CH1_{-other sequence-}AADC_{-APU-}TH; AADC_{-APU-}TH_{-APU-}CH1; AADC_{-APU-}TH_{-other sequence-}CH1; AADC_{-other sequence-}TH_{-APU-}CH1; AADC_{-APU-}CH1_{-APU-}TH; AADC_{-APU-}CH1_{-other sequence-}TH; or AADC_{-other sequence-}CH1_{-APU-}TH, wherein the other sequence comprises a linker peptide coding sequence, an internal ribosome entry site (IRES), a promoter, or an intein coding sequence.

17. The gene sequence construct of any of the preceding embodiments, further comprising a promoter.

18. The gene sequence construct of embodiment 17, wherein the promoter is a constitutive promoter selected from the group of a CMV promoter, a CBH promoter, a phosphoglycerate kinase promoter, and a thymidine kinase promoter.

19. The gene sequence construct of embodiment 17, wherein the promoter is a tissue-specific promoter selected from the group consisting of a synapsin promoter, a CD68 promoter, a GFAP promoter, and a synthetic promoter.

20. A viral vector comprising the gene sequence construct of any of embodiments 1-19.

21. The viral vector of embodiment 20, wherein the viral vector is a lentiviral vector or adeno-associated viral vector.

22. A viral vector genome comprising the gene sequence construct of any of embodiments 1-19.

23. The viral vector genome of embodiment 22, wherein the viral vector genome is a lentiviral vector genome or adeno-associated viral vector genome.

24. A viral vector system comprising the gene sequence construct of any of embodiments 1-19.

25. The viral vector system of embodiment 24, wherein the viral vector system is a lentiviral vector system or adeno-associated viral vector system.

26. A viral particle produced by the viral vector of embodiment 20 or 21, or the viral vector system of embodiment 24 or 25.

27. A cell transduced by the viral vector of embodiment 20 or 21, or the viral vector system of embodiment 24 or 25.

28. A biological product comprising the gene sequence construct of any one of embodiments 1-19, the viral vector of embodiment 20 or 21, the viral vector genome of embodiment 22 or 23, the viral vector system of embodiment 24 or 25, the viral particle of embodiment 26, or the cell of embodiment 27.

29. A pharmaceutical composition comprising the gene sequence construct of any one of embodiments 1-19, the viral vector of embodiment 20 or 21, the viral vector genome of embodiment 22 or 23, the viral vector system of embodiment 24 or 25, the viral particle of embodiment 26, or the cell of embodiment 27, and a pharmaceutically acceptable excipient or carrier.

30. A method of treating or preventing a neurodegenerative disease, comprising administering to a subject in need thereof an effective amount of the viral vector of embodiment 20 or 21, the viral vector system of embodiment 24 or 25, the viral particle of embodiment 26, the cell of embodiment 27, the biological product of embodiment 28, or the pharmaceutical composition of embodiment 29, thereby treating or preventing the neurodegenerative disease.

31. The method of embodiment 30, wherein the neurodegenerative disease comprises Parkinson's disease.

32. The method of embodiment 31, wherein the neurodegenerative disease comprises Alzheimer's disease.

33. The method of any of embodiments 30-32, wherein the subject is a human.

34. A method of producing dopamine, comprising contacting a cell with the viral vector of embodiment 20 or 21, the viral vector system of embodiment 24 or 25, the viral particle of embodiment 26, the cell of embodiment 27, the biological product of embodiment 28, or the pharmaceutical composition of embodiment 29, thereby producing dopamine.

35. The method of embodiment 34, wherein the cell is a human cell.

36. The method of embodiment 34 or 35, wherein the contacting occurs in vivo or eA vivo.

The present invention is further described in detail below in conjunction with examples. The following examples explain the present invention and the present invention is not limited to the following examples.

### EXAMPLES

### Example 1

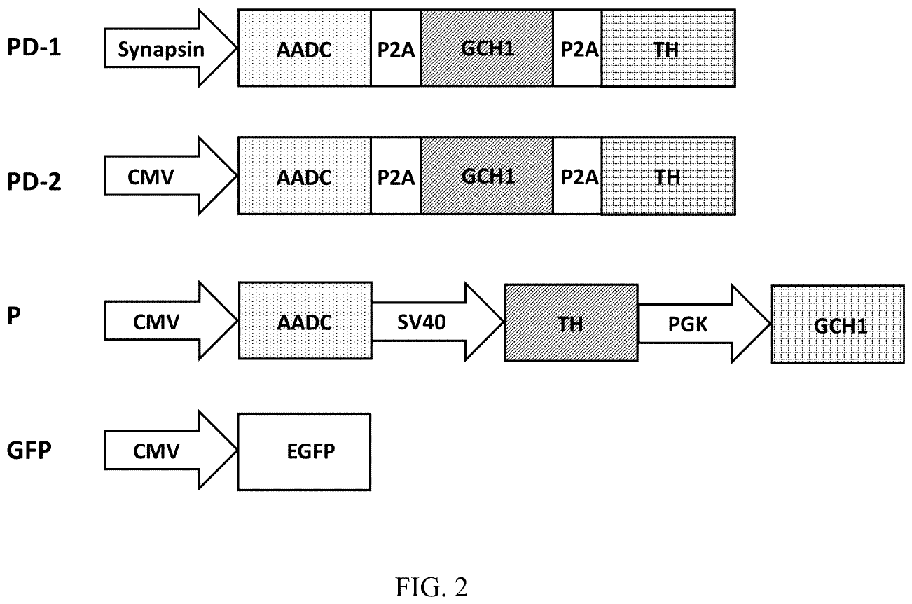

### I. The Construction of Various Constructs as Shown in FIG. 2:

KL0039 vector, synthetic CMV enhancer-synapsin promoter-AADC-P2A-GCH1-P2A-TH and AADC-SV40 promoter-TH-PGK promoter-GCH1 sequences (where TH is a truncated form of TH); wherein, CMV enhancer-synapsin promoter-AADC-P2A-GCH1-P2A-TH is ligated into pUC57 vector (pUC57-synapsin-AGT). Here, the KL0039 vector is a lentiviral transfer vector, derived from existing lentiviral vectors with partial modifications as needed.

### 1. Construction of PD1 Vector

A PCR product was amplified from the sequence from WPRE to cPPT using KL0039 as template and primers Age-F and Sal-R and purified after electrophoresis. The primer sequences are: Age-F, CTGAGTGCCATTGGATGAcaatcaacctctggattaca (SEQ ID NO: 26); Sal-R, gattactattaataactactcacgcatgctcttctcca (SEQ ID NO: 27). Plasmid pUC57-synapsin-AGT was digested with AgeI and SalI, and the 4.1-kb fragment was recovered. The ligation products of the purified PCR product and synapsin-AGT fragment by T4 DNA ligase were used to transform DH5α competent cells. Transformant colonies were screened by PCR and the positive clones were further confirmed by sequencing.

### 2. Construction of PD2 Vector

Using KL0039 vector as template and primers SnaBI-F:

TCAGtacgtattagtcatcgctat (SEQ ID NO: 29) and SpeI-R:

CGATactagtgagctctgcttatataga (SEQ ID NO: 30), a PCR product (245 bp) of CMV promoter was amplified and purified after electrophoresis. Double digestion by SnaBI and SpeI was performed on the purified PCR product of CMV promoter and plasmid pUC57-synapsin-AGT, respectively, and the fragments of CMV promoter and pUC57-AGT were purified from the digestion products. The ligation product of the two fragments by T4 DNA ligase was used to transform DH5α competent cells. Transformant colonies were screened by PCR and the positive clones were further confirmed by sequencing. The positive clone was named pUC57-CMV-AGT. Then, a PCR product was amplified from the sequence from WPRE to cPPT using KL0039 as template and Age-F+Sal-R and purified after electrophoresis. Double digestion by AgeI and SalI was performed on the purified PCR product and plasmid pUC57-CMV-AGT, respectively, and the fragments of the PCR product and CMV-AGT were purified from the digestion products. The ligation product of the two fragments by T4 DNA ligase was used to transform DH5α competent cells. Transformant colonies were screened by PCR and the positive clones were further confirmed by sequencing.

3. Construction of P vector: the AADC-SV40 promoter-TH-PGK promoter-GCH1 sequence was used to replace the AGT sequence in PD2 vector.

4. GFP vector: the EGFP sequence was cloned and used to replace the AGT sequence in PD2 vector.

II. Evaluation of the Differential Expression of Target Proteins in 293T and SH-SY5Y Cells after Transduction with Various Constructs

Lentiviral four-plasmid system was used to transiently transfect 293T cell line, packaging GFP (CMV promoter-EGFP), PD1 (synapsin promoter-AGT), PD2 (CMV promoter-AGT) lentivirus and positive control virus P (CMV promoter-AADC-SV40 promoter-TH-PGK promoter-GCH1), respectively. The initial viruses were concentrated after purification and transduced into 293T cells after dilution. The titers were determined using RT-PCR (WPRE/ALB). The vector titers of all constructs were similar, ranging from 3.4E+09 TU/ml to 8.74E+09 TU/ml.

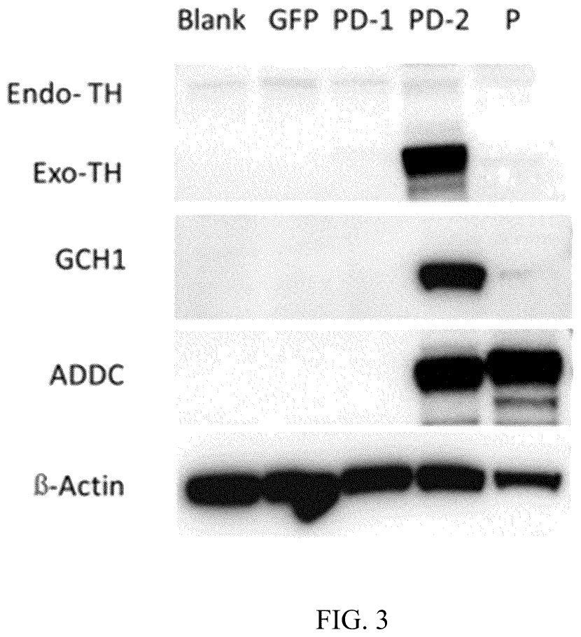

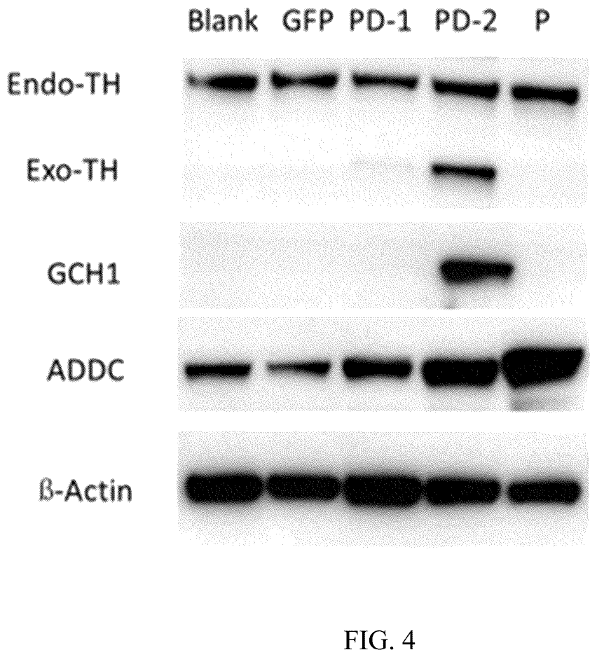

In order to assess the expression levels of target proteins, 293T cells and SH-SY5Y cells were transduced with the lentiviruses at MOI=10 and MOI=20, respectively. The cells were harvested 72 hours after transduction and cell lysates were used for Western blot analysis of AADC, GCH1, and TH. The results show that a relatively low level of endogenous TH, but no endogenous AADC and GCH1, was detected in 293T cells. The molecular weights of all target proteins were consistent with the expected values. Compared with no virus transduction Blank and GFP virus transduction, high levels of expression of all three target proteins were detected from cells transduced with PD2 viral vector. Although cells transduced with P viral vector expressed the highest level of AADC, the other two target proteins GCH1 and TH were barely detected. As expected, no expression of the three target proteins was detected in 293T cells transduced with PD1 viral vector, in which the synapsin promoter used is neuron-specific (FIG. 3). Further, we assessed the expression of targeted proteins in SH-SY5Y cells after transduction with the three different constructs. Endogenous TH and AADC with expected molecular weights were detected, but not endogenous GCH1. Similar to the results from transduced 293T cells, high levels of expression of all three target proteins were detected from cells transduced with PD2 viral vector, when compared with no virus transduction Blank and GFP virus transduction. Although cells transduced with P viral vector expressed the highest level of AADC, the other two proteins GCH1 and TH were still barely detected. For cells transduced with PD1 viral vector, only low levels of exogenous AADC and TH were expressed, and GCH1 was barely detected (FIG. 4).

III. Evaluation of the Differential Catecholamine Production in SH-SY5Y Cells after Transduction with Various Constructs

In neurons, DA is converted primarily by monoamine oxidase (MAO) to dihydroxyphenylacetic acid (DOPAC). The levels of catecholamine were measured by mass spectrometry in the supernatants of two cultured cells, SH SY5Y cells and 293T cells, after transduction with lentiviral vectors. The SH SY5Y cells were transduced by viruses and the media were replaced after overnight. The supernatants were collected after being cultured until the third day and centrifuged at 4500 rpm for 5 minutes. The clear supernatants were transferred to 1.5 mL centrifugation tubes and stored in freezer at −80° C. before testing. The 293T cells were transduced by viruses and the media were changed after overnight. After 2 days of culture, the cells were passaged at 1:10. After 2 days of culture, the media were replaced with fresh media containing 10 mM L-tyrosine. The supernatants were collected after being cultured until the next morning and centrifuged at 4500 rpm for 5 min. The clear supernatants were transferred to 1.5 mL centrifuge tubes and stored in freezer at −80° C. before testing.

The levels of dopamine in the samples were measured by mass spectrometry as follows. 500 μL of cell culture medium was collected and an appropriate amount of internal standard was added. The solution was diluted and mixed with 1 mL of 50 mM ammonium acetate serving as the sample loading solution. After methanol activation, the cartridge was subsequently rinsed with 20 mM ammonium acetate, acetonitrile:isopropanol (1:1), and drained. The sample was eluted with 2% formic acid in acetonitrile and blown dry with nitrogen. The residue was dissolved in 100 μL of 0.1% FA and centrifuged at 15000 r/min for 5 min. The supernatant was loaded onto the machine (Agilent 1290UPLC-6470MS/MS detection system) for analysis.

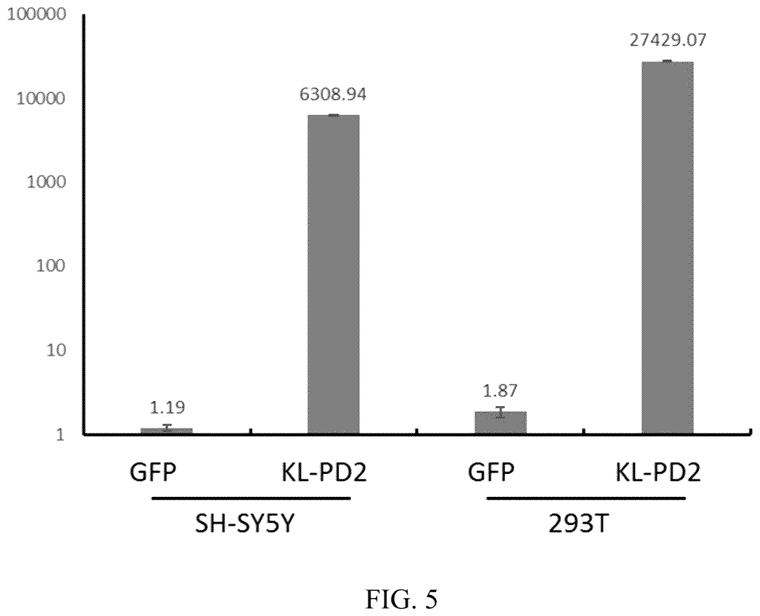

The results are shown in FIG. 5. Transduction of 293T cells and SH-SY5Y cells with KL-PD2 lentiviral vector greatly increased the production of dopamine in the cell culture supernatants due to the effective and balanced expression of the three enzymes in dopamine synthesis.

### Example 2

This example describes the use of gene sequence construct to quantify the increase of dopamine production both in vitro and in vivo. Striatal cells from mouse embryonic brains were isolated, cultured in vitro, and transduced with lentiviral (LV) or adeno-associated virus (AAV) vectors packaged with (1) EGFP expression vector as a negative control, (2) a claimed construct comprising AADC-P2A-GCH1-P2A-TH under a constitutive promoter (e.g., CMV promoter or CBH promoter), or (3) a claimed construct comprising AADC-P2A-GCH1-P2A-TH under the tissue-specific synapsin promoter. After 7 days of transduction, the cell culture supernatants were collected, and the levels of dopamine and its metabolites were determined by HPLC. In addition, to demonstrate the in vivo efficacy of the constructs, a rat model of Parkinson's disease was used in which unilateral dopamine depletion was induced by injection of 6-OHDA into the medial forebrain bundle. The rats were then injected stereotaxically with AAV vectors comprising the claimed construct (AADC-P2A-GCH1-P2A-TH) or control construct (EGFP). Four weeks after injection, the rats were assessed for contralateral rotational behavior induced by apomorphine.

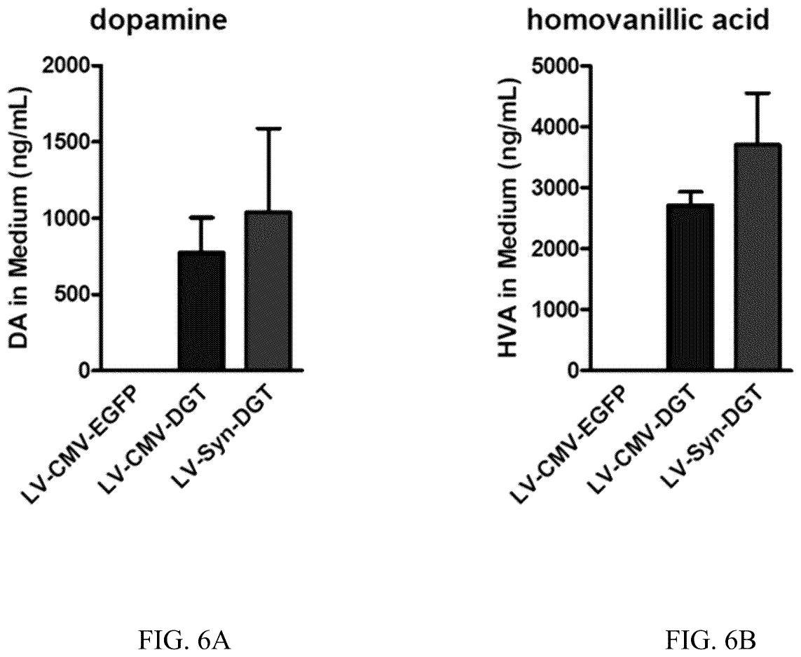

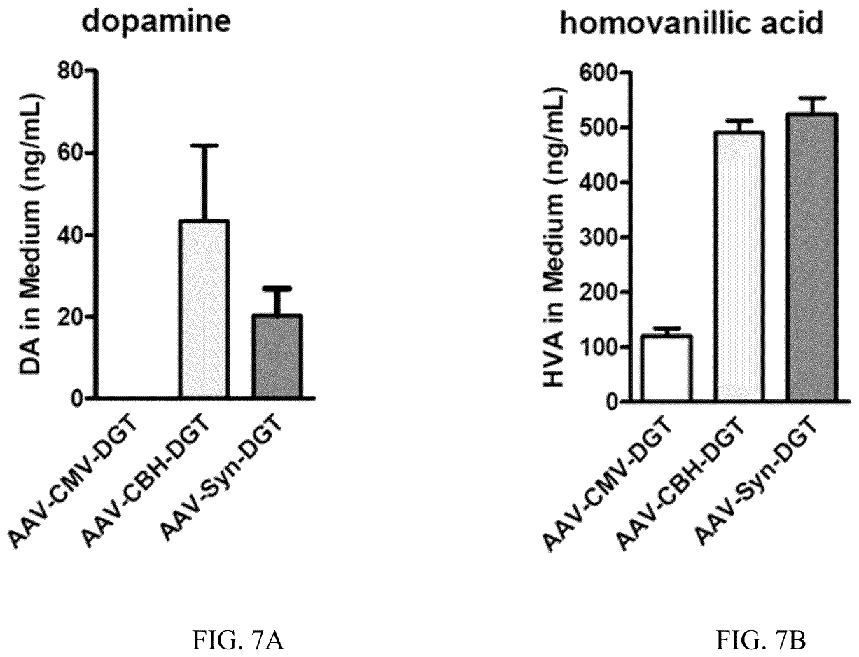

As shown in FIGS. 6A-6B, transduction of striatal cells with LV vectors comprising the claimed construct resulted in production of high levels of dopamine (mean>774 ng/mL) as well as elevated levels of the dopamine metabolite homovanillic acid (mean>2713 ng/mL). Similarly, transduction of striatal cells with AAV vectors comprising the claimed construct resulted in production of high levels of dopamine (above 43 ng/mL observed) and homovanillic acid (above 490 ng/mL observed), as shown in FIGS. 7A-7B.

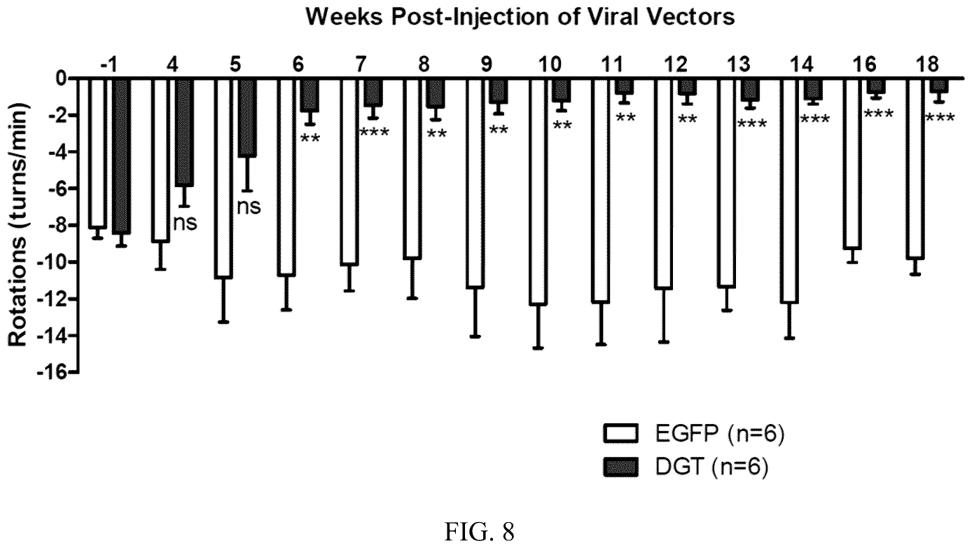

As shown in FIG. 8, administration of the AAV vector comprising the claimed construct resulted in significant recovery in contralateral rotational behavior as compared to the control vector, with an improvement of up to 84% observed.

### Example 3

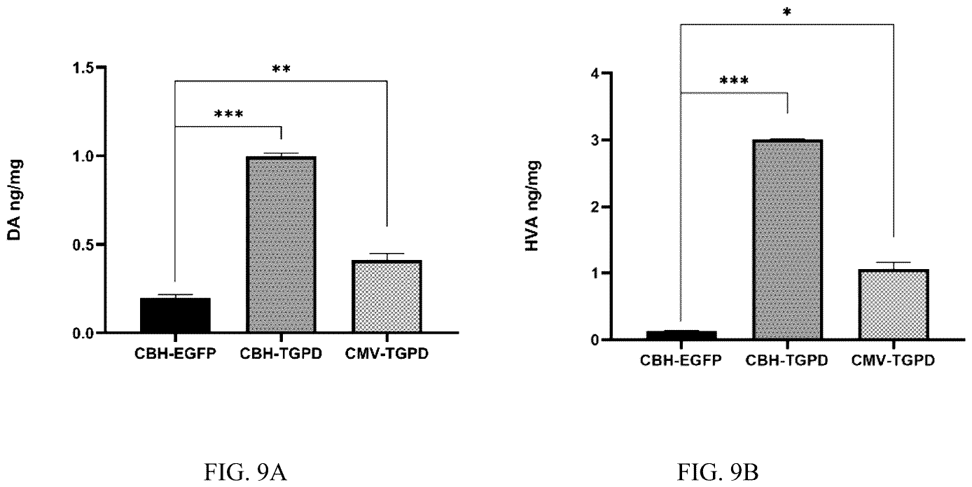

This example describes the use of gene sequence construct comprising different promoters to quantify the increase of dopamine production. Viral particles packaged with vectors using different promoters were delivered into the brains of wild-type C57/B6 mice via stereotactic injection at the same dose to infect neural cells. Two weeks post-injection, brain tissues were harvested, and for every 30 mg of brain tissue, 200 μL of a solution containing 0.2 mM HClO₄ and 1 mM cysteine was added. The tissues were homogenized to prepare cell lysates, which were then centrifuged at 12,000 rpm for 5 minutes at 4° C. The supernatant was collected, and dopamine concentration in the cell lysates was measured using high-performance liquid chromatography (HPLC). The experimental results (see Table 1 and FIG. 9) indicate that, compared to the control group, vectors with different promoters efficiently synthesized dopamine in vivo after infecting neural cells.

TABLE 1
In Vivo dopamine and homovanillic acid expression
levels with vectors having different promoters

| | Dopamine | Homovanillic Acid |
|---|---|---|
| Group | ng/mg | ng/mg |
| CBH-EGFP | 0.20 | 0.14 |
| (negative control) | 0.23 | 0.14 |
| | 0.16 | 0.13 |
| CBH-TGPD | 1.02 | 3.01 |
| (TH1-GS15-GCH1- | 1.01 | 3.03 |
| P2A-AADC) | 0.96 | 3.00 |
| CMV-TGPD | 0.49 | 1.24 |
| (TH1-GS15-GCH1- | 0.36 | 0.91 |
| P2A-AADC) | 0.38 | 1.05 |

### Example 4

### 1. Vector Construction

Two synthetic nucleotide sequences, TH1-P2A-GCH1-P2A-AADC (referred to as “TGD test construct” hereafter) and AADC-P2A-TH1-P2A-GCH1 (referred to as “DTG reference construct” hereafter), were constructed, in which TH1 is in a truncated form. The TGD test construct and the DTG reference construct were cloned into an adeno-associated virus (AAV) backbone, pAAV-MCS-CBH-SV40, between CBH and SV40 by recombinant ligation to prepare recombinant adeno-associated virus vectors (rAAV). The resulting vectors were named CBH-TGD vector and CBH-DTG vector, respectively.

### 2. AAV Virus Packaging and Assay

GBH-TGD vector and CBH-DTG vector were each purified and packaged into AAV using a serotype packaging plasmid, KL-pAAV9, and a helper plasmid, KL-pAAV-Helper, resulting in AAV viral particles AAV9-CBH-TGD and AAV9-CBH-DTG. The titers of the AAV9-CBH-TGD and AAV9-CBH-DTG were 1.15×10¹³ viral genomes per ml (vg/ml) and 1.45×10¹³ vg/ml, respectively.

### 3. Comparison of Dopamine Synthesis Levels In Vitro

Each of AAV9-CBH-TGD and AAV9-CBH-DTG were used to infect 293T cells at multiplicity of infection (MOI)=of 5.0×10⁵ (Group 1) and MOI=1.0×10⁶ (Group 2). The levels of dopamine were measured for each group in triplicate. Samples were centrifuged at 12,000 rpm for 5 min to collect cell supernatants. 50 ul of protective solution (1 M perchloric acid and 15 mM cysteine) was added to the cell supernatant of 200 uL, and the dopamine concentration was detected by high performance liquid chromatography.

### 4. Results

The levels of dopamine produced by the TGD test construct and the DTG reference construct at the two different multiplicities of infection are shown in Table 2 below.

TABLE 2
Dopamine levels in 293T cells infected
with AAV9-CBH-TGD or AAV9-CBH-DTG

| | | | Average Dopamine |
|---|---|---|---|
| | | Dopamine | Level ± Standard |
| Construct | MOI | (ng/mL) | Deviation (ng/mL) |
| CBH-TGD | 5.0 × 10⁵ | 1271.1 | 1608.7 ± 294.3 |
| | | 1753.0 | |
| | | 1801.8 | |
| CBH-DTG | | 755.1 | 711.9 ± 60.5 |
| | | 737.8 | |
| | | 642.8 | |
| CBH-TGD | 1.0 × 10⁶ | 4805.4 | 5166.3 ± 325.7 |
| | | 5438.4 | |
| | | 5255.2 | |
| CBH-DTG | | 2223.3 | 2103.3 ± 201.8 |
| | | 2216.3 | |
| | | 1870.3 | |

As shown in Table 2, the TGD test construct resulted in a significantly higher efficiency in dopamine synthesis compared to the DGT reference construct.

### Example 5

This example describes the use of gene sequence construct comprising different 2A peptides to quantify the increase of dopamine production. Synthetic nucleotide sequence TH-F2A-GCH1-P2A-AADC (referred to as “TFGPD construct” hereafter) was constructed and cloned into an adeno-associated virus (AAV) backbone, pAAV-MCS-CBH-SV40, between CBH and SV40 by recombinant ligation to prepare recombinant adeno-associated virus vectors (rAAV). The resulting vector was named CBH-TFGPD vector. CBH-EGFP vector, which was constructed similarly, was used as negative control.

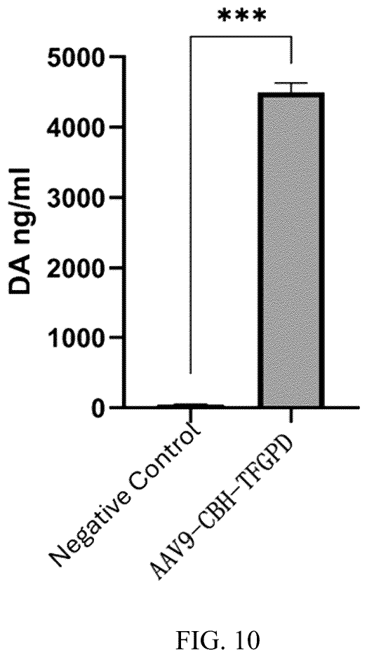

Packaged AAV viral particles were used to infect 293T cells. After 72 hours, the levels of dopamine and its metabolites in the cell supernatant collected were measured by high-performance liquid chromatography. Table 3 and FIG. 10 show that after AAV9-CBH-TFGPD infection in vitro, intracellular dopamine synthesis significantly increased. The levels of 3,4-dihydroxyphenylacetic acid and homovanillic acid (which are dopamine metabolites) also significantly increased, demonstrating that CBH-TFGPD construct resulted in efficient dopamine synthesis in vitro.

TABLE 3
AAV vector-mediated in vitro dopamine
synthesis and metabolite detection

| | | 3,4-Dihydroxy- | | |
|---|---|---|---|---|
| | | phenylacetic | Homovanillic | |
| AAV | | Acid | Acid | Dopamine |
| Vector | MOI | Concentration (ng/mL) | | |
| CBH-EGFP | 5.00E+05 | 75.8 | 186.7 | 37.1 |
| | | 76.1 | 174.6 | 49.1 |
| | | 76.2 | 175.8 | 49.2 |
| CBH-TFGPD | | 340.3 | 845.9 | 4394.0 |
| | | 305.7 | 807.6 | 4448.5 |
| | | 318.4 | 768.2 | 4650.8 |

In addition, it should be noted that the specific examples described in this specification may bear different names for various substances or carriers. Any equivalent or simple variation made according to the structure configuration and principles described in the patent conception of the present invention all belong to the scope of the present patent protection. Those skilled in the art to which the present invention pertains can make various modifications or additions to the described specific examples or substitute in a similar manner, as long as they do not depart from the structures of the present invention or go beyond the scope defined by the claims, all should belong to the scope of protection of the present invention.

1. A gene sequence construct comprising a plurality of nucleotide sequences comprising the nucleotide sequences of human tyrosine hydroxylase (TH), GTP-cyclohydrolase I (GCH1), and aromatic amino acid decarboxylase (AADC), wherein two or more of the nucleotide sequences of human TH, GCH1, and AADC are linked by a nucleotide sequence encoding a 2A peptide, and wherein expression of the plurality of nucleotide sequences promotes synthesis of dopamine.

2. The gene sequence construct of claim 1, wherein all of the nucleotide sequences of human TH, GCH1, and AADC are linked by a nucleotide sequence encoding a 2A peptide.

3. The gene sequence construct of claim 1, wherein the 2A peptide is selected from the group consisting of a 2A peptide from foot-and-mouth disease virus (F2A), a 2A peptide from porcine teschovirus (P2A), a 2A peptide from insect virus (T2A), and a 2A peptide from equine rhinitis virus (E2A).

4. The gene sequence construct of claim 1, wherein the gene sequence construct comprises the following modes of construction: TH_{-2A-}GCH1_{-2A-}AADC; TH_{-2A-}GCH1_{-other sequence-}AADC; TH_{-other sequence-}GCH1_{-2A-}AADC; TH_{-2A-}AADC_{-2A-}GCH1; TH_{-other sequence-}AADC_{-2A-}GCH1; TH_{-2A-}AADC_{-other sequence-}GCH1; GCH1_{-2A-}TH_{-2A-}AADC; GCH1_{-2A-}TH_{-other sequence-}AADC; GCH1_{-other sequence-}TH_{-2A-}AADC; GCH1_{-2A-}AADC_{-2A-}TH; GCH1_{-2A-}AADC_{-other sequence-}TH; GCH1_{-other sequence-}AADC_{-2A-}TH; AADC_{-2A-}TH_{-2A-}GCH1; AADC_{-2A-}TH_{-other sequence-}GCH1; AADC_{-other sequence-}TH_{-2A-}GCH1; AADC_{-2A-}GCH1_{-2A-}TH; AADC_{-2A-}GCH1_{-other sequence-}TH; or AADC_{-other sequence-}GCH1_{-2A-}TH, wherein the other sequence comprises a linker peptide coding sequence or an internal ribosome entry site (IRES).

5. The gene sequence construct of claim 1, further comprising a promoter.

6. The gene sequence construct of claim 5, wherein the promoter is a constitutive promoter.

7. The gene sequence construct of claim 6, wherein the constitutive promoter is selected from the group of a CMV promoter, a CBH promoter, a phosphoglycerate kinase promoter, and a thymidine kinase promoter.

8. The gene sequence construct of claim 5, wherein the promoter is a tissue-specific promoter.

9. The gene sequence construct of claim 8, wherein the tissue-specific promoter is selected from the group consisting of a synapsin promoter, a CD68 promoter, and a GFAP promoter.

10. The gene sequence construct of claim 1, wherein the nucleotide sequence of human TH comprises a nucleotide sequence encoding the amino acid sequence of SEQ ID NO: 11 or an amino acid sequence having at least 90% identity thereto.

11. The gene sequence construct of claim 1, wherein the nucleotide sequence of human GCH1 comprises a nucleotide sequence encoding the amino acid sequence of SEQ ID NO: 9 or an amino acid sequence having at least 90% identity thereto.

12. The gene sequence construct of claim 1, wherein the nucleotide sequence of human AADC comprises a nucleotide sequence encoding the amino acid sequence of SEQ ID NO: 7 or an amino acid sequence having at least 90% identity thereto.

13. A viral vector comprising the gene sequence construct of claim 1.

14. The viral vector of claim 13, wherein the viral vector is a lentiviral vector.

15. The viral vector of claim 13, wherein the viral vector is an adeno-associated viral vector.

16. A pharmaceutical composition comprising the viral vector of claim 13 and a pharmaceutically acceptable carrier.

17. A cell transduced by the viral vector of claim 13.

18. A viral particle produced by the viral vector of claim 13.

19. A method of treating a neurodegenerative disease, comprising administering to the subject in need thereof an effective amount of the viral vector claim 16, thereby treating Parkinson's disease.

20. A method of producing dopamine, comprising contacting a cell in a subject with the viral vector of claim 16, thereby producing dopamine.


SEQUENCE LISTING (30 sequences)

SEQ ID NO: 1 - DNA, 814 bp, unidentified
  source 1..814: mol_type = unassigned DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggt

SEQ ID NO: 2 - DNA, 706 bp, unidentified
  source 1..706: mol_type = unassigned DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcg

SEQ ID NO: 3 - DNA, 580 bp, unidentified
  source 1..580: mol_type = unassigned DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcag

SEQ ID NO: 4 - DNA, 897 bp, unidentified
  source 1..897: mol_type = unassigned DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgt

SEQ ID NO: 5 - DNA, 1601 bp, unidentified
  source 1..1601: mol_type = unassigned DNA
gttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 6 - DNA, 1052 bp, unidentified
  source 1..1052: mol_type = unassigned DNA
gttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagc

SEQ ID NO: 7 - protein, 480 aa, Homo sapiens
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 8 - DNA, 1440 bp, Homo sapiens
  source 1..1440: mol_type = genomic DNA
atgaacgcaagtgaattccgaaggagagggaaggagatggtggattacatggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggag

SEQ ID NO: 9 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 10 - DNA, 750 bp, Homo sapiens
  source 1..750: mol_type = genomic DNA
atggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagc

SEQ ID NO: 11 - protein, 338 aa, Homo sapiens
MVKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 12 - DNA, 1014 bp, Homo sapiens
  source 1..1014: mol_type = genomic DNA
atggtgaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattgga

SEQ ID NO: 13 - protein, 22 aa, synthetic construct
GSGATNFSLLKQAGDVEENPGP

SEQ ID NO: 14 - DNA, 66 bp, synthetic construct
  source 1..66: mol_type = other DNA
ggaagcggagctactaacttcagcctgctgaagcaggctggcgacgtggaggagaaccctggacct

SEQ ID NO: 15 - protein, 25 aa, synthetic construct
GSGVKQTLNFDLLKLAGDVESNPGP

SEQ ID NO: 16 - DNA, 75 bp, synthetic construct
  source 1..75: mol_type = other DNA
ggaagcggagtgaaacagactttgaattttgaccttctcaagttggcgggagacgtggagtccaaccctggacct

SEQ ID NO: 17 - protein, 15 aa, synthetic construct
GGGGSGGGGSGGGGS

SEQ ID NO: 18 - DNA, 45 bp, synthetic construct
  source 1..45: mol_type = other DNA
ggaggcggcggctctggtggaggcggcagcggcggcggcggttct

SEQ ID NO: 19 - protein, 21 aa, synthetic construct
GSGEGRGSLLTCGDVEENPGP

SEQ ID NO: 20 - DNA, 63 bp, synthetic construct
  source 1..63: mol_type = other DNA
ggaagcggagagggcagaggaagtctgctaacatgcggtgacgtcgaggagaatcctggccca

SEQ ID NO: 21 - protein, 23 aa, synthetic construct
GSGQCTNYALLKLAGDVESNPGP

SEQ ID NO: 22 - DNA, 69 bp, synthetic construct
  source 1..69: mol_type = other DNA
ggaagcggacagtgtactaattatgctctcttgaaattggctggagatgttgagagcaaccctggacct

SEQ ID NO: 23 - DNA, 858 bp, unidentified
  source 1..858: mol_type = unassigned DNA
ctcaaacccgctgatcagcctcgactgtgccttctagttgccagccatctgttgtttgcccctcccccgtgccttccttgagaattcctcgacgtagatatcttaaaacagctctggggttgtacccaccccagaggcccacgtggcggctagtactccggtattgcggtacctttgtacgcctgttttatactcccttcccccgtaacttagaagcacaatgtccaagttcaataggagggggtgcaaaccagtaccaccacgaacaagcacttctgttcccccggtgaggctgtataggctgtttccacggctaaaagcggctgatccgttatccgctcatgtacttcgagaagcctagtatcaccttggaatcttcgatgcgttgcgctcaacactcaaccccagagtgtagcttaggtcgatgagtctggacgttcctcaccggcgacggtggtccaggctgcgttggcggcctacctgtggcccaaagccacaggacgctagttgtgaacaaggtgtgaagagcctattgagctacctgagagtcctccggcccctgaatgcggctaatcctaaccacggagcaggcagtggcaatccagcgaccagcctgtcgtaacgcgcaagttcgtggcggaaccgactactttgggtgtccgtgtttccttttatttttacaatggctgcttatggtgacaatcattgattgttatcataaagcaaattggattggccatccggtgagaatttgattattaaattactctcttgttgggattgctcctttgaaatcttgtgcactcacacctattggaattacctcattgttaagatacgcgtctagctagcgccacc

SEQ ID NO: 24 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 25 - DNA, 636 bp, Homo sapiens
  source 1..636: mol_type = genomic DNA
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 26 - DNA, 38 bp, synthetic construct
  source 1..38: mol_type = other DNA
ctgagtgccattggatgacaatcaacctctggattaca

SEQ ID NO: 27 - DNA, 38 bp, synthetic construct
  source 1..38: mol_type = other DNA
gattactattaataactactcacgcatgctcttctcca

SEQ ID NO: 28 - protein, 30 aa, synthetic construct
  VARIANT 1..30: note = This sequence may encompass 1-6 GGGGS repeating units
GGGGSGGGGSGGGGSGGGGSGGGGSGGGGS

SEQ ID NO: 29 - DNA, 24 bp, synthetic construct
  source 1..24: mol_type = other DNA
tcagtacgtattagtcatcgctat

SEQ ID NO: 30 - DNA, 28 bp, synthetic construct
  source 1..28: mol_type = other DNA
cgatactagtgagctctgcttatataga